(19) United States
(12) Patent Application Publication
(10) Pub. No.: US 2023/0348869 A1
(43) Pub. Date: Nov. 2, 2023

(54) MAD7 NUCLEASE IN PLANTS AND EXPANDING ITS PAM RECOGNITION CAPABILITY

(71) Applicant: KWS SAAT SE & Co. KGaA, Einbeck (DE)

(72) Inventors: Zarir VAGHCHHIPAWALA, Ballwin, MO (US); Yu MEI, Olivette, MO (US)

(73) Assignee: KWS SAAT SE & Co. KGaA, Einbeck (DE)

(21) Appl. No.: 17/768,635

(22) PCT Filed: Oct. 14, 2020
(86) PCT No.: PCT/EP2020/078845

§ 371 Date: Apr. 13, 2022

Related U.S. Application Data
(60) Provisional application No. 62/914,825, filed on Oct. 14, 2019.

(51) Int. Cl. C12N 9/22; C12N 15/11; C12N 15/82

(52) U.S. Cl. CPC C12N 9/22; C12N 15/11; C12N 15/8213; C12N 2310/20

(57) ABSTRACT

The present invention relates to a MAD7-type nuclease, which has been engineered to recognize a PAM selected from TYCV, TATV or TTCN. The invention provides sequences encoding or representing a MAD7-type nuclease carrying certain mutations compared to the sequence of a MAD7 nuclease. The invention also provides a genome engineering system, an expression construct and a kit comprising a MAD7-type nuclease according to the invention. Moreover, the invention relates to a method for the targeted modification of at least one genomic target sequence in a cell, which comprises introducing the MAD7-type nuclease according to the invention into the cell. The invention also provides a cell and an organism obtained by a method according to the invention.

### CROSS-REFERENCE TO RELATED APPLICATIONS

This application is a U.S. National Phase of International Pat. Application No. PCT/EP2020/078845, filed on Oct. 14, 2020, which claims priority to U.S. Application No. 62/914,825, filed Oct. 14, 2019. The entire contents of these applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 26, 2023, is named 245761_000166_SL.txt and is 368,541 bytes in size.

### TECHNICAL FIELD

The present invention relates to a MAD7-type nuclease, which has been engineered to recognize a PAM selected from TYCV, TATV or TTCN. The invention provides sequences encoding or representing a MAD7-type nuclease carrying certain mutations compared to the sequence of a MAD7 nuclease. The invention also provides a genome engineering system, an expression construct and a kit comprising a MAD7-type nuclease according to the invention. Moreover, the invention relates to a method for the targeted modification of at least one genomic target sequence in a cell, which comprises introducing the MAD7-type nuclease according to the invention into the cell. The invention also provides a cell and an organism obtained by a method according to the invention. Further provided are a method of producing a chimeric MAD7-type nuclease and a method of treating a disease in a subject using the MAD7-type nuclease according to the invention.

### BACKGROUND

Nucleic acid guided nucleases (NGNs) have emerged as promising and reliable tools in genome engineering/editing of prokaryotic and eukaryotic genomes over the last decade. In particular, CRISPR nucleases have been the focus of large developments due to the fact that they can readily be programmed to introduce a double strand break at a specific position of a sequence of interest in a range of cells.

In view of the fact that eukaryotic genomes, for example the genomes of fungi, plants, animals and humans, are rather diverse regarding complexity and codon usage, however, there are still strong limitations associated with certain NGNs. One aspect is the off-target activity of a given nucleic acid guided nuclease which will be different in different cells to be modified. Therefore, efficiency may vary significantly from one setting to the next. The most critical limiting factor in transferring the activity of a given nucleic acid guided nuclease to a broad spectrum of eukaryotic cells is the intrinsic protospacer adjacent motif (PAM) specificity of a nucleic acid guided nuclease. Due to this specificity, the target sequence has to be accompanied by a specific PAM to be recognized and cleaved by the nuclease.

The PAM is a short DNA sequence (about 2 to 6 base pairs long), which is located a few nucleotides from the cut site of the nuclease. The most commonly used Cas9 nuclease from Streptococcus pyogenes recognizes a 5′-NGG-3′ PAM. If such a motif is not present at the target site, there is a number of Cas9 nucleases from other organisms available, from which one with a more suitable PAM may be chosen. Still, the number of PAMs specificities available is limited.

Cpf1 nucleases provide advantages over Cas9 for some applications including the requirement of only one guide RNA molecule and the generation of sticky ends at the cut site, which facilitates the insertion of sequences. The Cpf1 nuclease of Lachnospiraceae bacterium (LbCpf1) and Acidaminococcus sp. (AsCpf1) both recognize a 5′-TTTV-3′ PAM. However, such T-rich PAMs are relatively rare in higher eukaryotic genomes, limiting the applicability of Cpf1 nucleases.

Gao et al. (Nat. Biotechnol. 2017, 35(8): 789-792) engineered Cpf1 RR and RVR variants with altered PAM specificities to increase the target range of Cpf1 in human coding sequences. Certain mutants were created, which recognized TYCV and TATV PAMs and showed enhanced activities in human cells. A similar approach was taken by Tóth et al. (Nucleic Acids Research, 2018, Vol. 46, No. 19, 10272-10285). They generated corresponding Fn- and McCpf1 mutants, which gained new PAM specificities but also retained their activity on targets with TTTV PAMs.

MAD7, a CRISPR class II type V nuclease, was initially isolated from Eubacterium rectale and re-engineered by Inscripta. The nuclease is disclosed to have a 5′-YTTN-3′ (i.e., CTTN or TTTN) PAM specificity, i.e. these PAMs provide the highest editing efficiency (WO2018236548A1). Gene editing activity for MAD7 was demonstrated in E. coli and yeast but also in mammalian cells. It was also shown that MAD7 can be used for a targeted knockout of the CPL3 gene in maize (WO2020/178215).

There is still a great need to expand the targeting range for NGNs to have a full genome coverage for complex plant, animal and human genomes. However, the broadened applicability has to be achieved without sacrificing efficiency or specificity of the nucleic acid guided nuclease system (NGN + guide RNA).

It was an objective of the present invention to provide engineered nucleases, which have an altered or broadened targeting range with respect to the original nuclease. In particular, the PAM specificity of the nucleases should be altered or broadened to allow new applications in genome engineering. On the other hand, target specificity and overall activity should remain at least comparable to the original nuclease.

### SUMMARY OF THE INVENTION

In a first aspect, the present invention provides a nucleic acid guided nuclease, wherein the nuclease is a MAD7-type nuclease, or a sequence encoding the same, with an engineered PAM specificity, wherein the nuclease is engineered to recognize at least one PAM selected from the group consisting of TYCV, TATV, or TTCN.

In one embodiment of the various aspects of the present invention, the nuclease additionally recognizes at least one PAM selected from the group consisting of TTTN and CTTN.

In another embodiment of the various aspects of the present invention, the nuclease has a higher activity on a CTTN site in comparison to a MAD7 nuclease.

In a further embodiment of the various aspects of the present invention, the nuclease, or a domain thereof, comprises at least one mutation at position 177, 272, 537, 543, 547, or 602 in comparison to the reference sequence of the MAD7 nuclease according to SEQ ID NO:3, or a combination of mutations.

In yet another embodiment of the various aspects of the present invention, the at least one mutation described above is independently selected from K177R, N272A, D537R, K543V, K543R, N547R, K602R, or a combination thereof.

In one embodiment of the various aspects of the present invention, the nuclease, or a domain thereof, comprises a combination of mutations selected from D537R+K602R (MAD7-RR), D537R+K543V+N547R (MAD7-RVR), K177R+D537R (MAD7-V1), K177R+D537R+K543R (MAD7-V2), K177R+D537R+K602R (MAD7-RRR), K177R+D537R+K543V+N547R (MAD7-RRVR), K177R+D537R+N272A (MAD7-V1 + N272A), K177R+D537R+N272A+K543R (MAD7-V2 + N272A), K177R+D537R+N272A+K602R (MAD7-RRR + N272A), K177R+D537R+K543V+N272A+N547R (MAD7-RRVR + N272A).

In another embodiment of the various aspects of the present invention, the nuclease comprises an amino acid sequence of any one of SEQ ID NOs: 12, 15, 18, 21, 24, 27, 30, 33, 36, 39, 40 or 41 or an amino acid sequence having at least 90%, 91%, 92%, 93%, 94%, 95%, 96%, 97%, 98% or 99% sequence identity to the respective amino acid sequence, or the nuclease is encoded by a nucleic acid sequence of any one of SEQ ID NOs: 10, 11, 13, 14, 16, 17, 19, 20, 22, 23, 25, 26, 28, 29, 31, 32, 34, 35, 37, 38 or a codon optimized variant thereof, or a nucleic acid sequence encoding an amino acid sequence having at least 90%, 91%, 92%, 93%, 94%, 95%, 96%, 97%, 98% or 99% sequence identity to any one of the respective SEQ ID NOs: 12, 15, 18, 21, 24, 27, 30, 33, 36, 39, 40 or 41.

In a further embodiment of the various aspects of the present invention, the nuclease comprises at least one mutation rendering the nuclease to a nickase or to a nuclease-dead variant of the nuclease, preferably the nuclease comprises a D885A and/or a E970A mutation or the nuclease comprises a R1181A mutation in comparison to the reference sequence of the MAD7 nuclease according to SEQ ID NO:3.

In one embodiment of the various aspects of the present invention, the nuclease comprises at least one nuclear localization signal, preferably the nuclease comprises one nuclear localization signal at the N-terminus and one nuclear localization signal at the C-terminus.

In another embodiment of the various aspects of the present invention, the nucleic acid sequence encoding the nuclease is codon optimized for expression in a target cell of interest.

In another aspect, the present invention provides a genome engineering system comprising at least one MAD7-type nuclease according to any of the embodiments described above, or a sequence encoding the same, and at least one guide nucleic acid sequence, or a sequence encoding the same, wherein the at least one guide nucleic acid sequence comprises a scaffold region and a targeting region.

In one embodiment of the genome engineering system described above, the targeting region targets a genomic target region of interest, which is an endogenous or isolated nucleic acid region of a eukaryotic cell.

In another embodiment of the genome engineering system according to any of the embodiments described above, the genomic target region of interest is an endogenous or isolated nucleic acid region of a plant cell or organism.

In a further embodiment of the genome engineering system according to any of the embodiments described above, the system additionally comprises at least one repair template, or a sequence encoding the same.

In one embodiment of the genome engineering system described above, the at least one repair template comprises or encodes a double- and/or single-stranded sequence.

In another embodiment of the genome engineering system described above, the at least one repair template comprises symmetric or asymmetric homology arms.

In a further embodiment of the genome engineering system described above, the at least one repair template comprises at least one chemically modified base and/or backbone.

In one embodiment of the genome engineering system according to any of the embodiments described above, the at least one MAD7-type nuclease, or the sequence encoding the same, and/or the at least one guide nucleic acid, or the sequence encoding the same, and/or optionally the at least one repair template, or the sequence encoding the same, are provided simultaneously, or one after another.

In another aspect, the present invention relates to an expression construct comprising or encoding at least one MAD7-type nuclease as described in any of the embodiments above, and/or at least one guide nucleic acid sequence as described above, and/or at least one repair template.

In one embodiment of the expression construct described above, the construct comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

In another embodiment of the expression construct described above, the regulatory sequence comprises or encodes at least one promoter selected from the group consisting of ZmUbi1, BdUbi10 (SEQ ID NO: 4), ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In a further embodiment of the expression construct described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In one embodiment of the expression construct according to any of the embodiments described above, the construct comprises or encodes a combination of a ZmUbi1 promoter (SEQ ID NO: 8) and a ZmUbi1 intron (SEQ ID NO: 9), a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In another embodiment of the expression construct according to any of the embodiments described above, the construct comprises or encodes at least one self-cleaving ribozyme, preferably at least one hammerhead ribozyme and/or a hepatitis-delta virus (HDV) ribozyme (WO 2019/138052).

In a further embodiment of the expression construct described above, the regulatory sequence comprises or encodes at least one terminator selected from the group consisting of nosT, a double 35S terminator, a ZmEf1 terminator, an AtSac66 terminator, an octopine synthase (ocs) terminator, or a pAG7 terminator, or a combination thereof.

In another aspect, the present invention provides a kit comprising, in separate form, at least one compartment comprising at least one MAD7-type nuclease as described in any of the embodiments above, or a sequence encoding the same, and optionally at least one guide nucleic acid sequence as defined above, or a sequence encoding the same, and optionally at least one repair template, or a sequence encoding the same, wherein the kit additionally comprises suitable reagents for each of the at least one compartment.

In a further aspect, the present invention relates to a method for the targeted modification of at least one genomic target sequence in a cell, wherein the method comprises the following steps:
(a) introducing into the cell
   (i) at least one MAD7-type nuclease, or a sequence encoding the same, as described in any of the embodiments above, and at least one guide nucleic acid sequence, or a sequence encoding the same, as defined above; or
   (ii) at least one genome engineering system as defined above or at least one expression construct as defined above,
   (iii) and, optionally at least one repair template, or a sequence encoding the same;
(b) cultivating the cell under conditions allowing the expression and/or assembly of the genome engineering system comprising the at least one MAD7-type nuclease and the at least one guide nucleic acid sequence and optionally the at least one repair template; and
(c) obtaining at least one modified cell.

In one embodiment of the method described above, (i), (ii), and optionally (iii) is/are introduced simultaneously or one after another.

In another embodiment of the method according to any of the embodiments described above, at least one of (i), (ii), and optionally (iii) is/are transiently introduced into and/or expressed in the cell.

In a further embodiment of the method described above, at least one of (i), (ii), and optionally (iii) is/are stably introduced into and/or expressed in the cell.

In one embodiment of the method according to any of the embodiments described above, at least two, three, four, five or more different guide nucleic acid sequences, or sequences encoding the same, are introduced into the cell to make multiple modifications in the cell simultaneously.

In another embodiment of the method according to any of the embodiments described above, the targeted modification of the at least one genomic target sequence in a cell is selected from at least one point mutation, at least one insertion, or at least one deletion, or any combination thereof.

In one embodiment of the method according to any of the embodiments described above, the cell is a eukaryotic cell, preferably a plant cell, an animal cell, a mammalian cell, or a human cell.

In another embodiment of the method according to any of the embodiments described above, the cell is a plant cell, which originates from a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Spinacia oleracea, Vicia faba, Phaseolus vulgaris, Allium cepa, Allium fistulosum, Allium sativum, and Allium tuberosum.

In one aspect, the present invention relates to a cell, preferably a eukaryotic cell selected from a plant cell, obtainable by a method as described in any of the embodiments above.

In another aspect, the present invention provides an organism, or part of an organism, preferably a plant, or part thereof, or a progeny thereof obtainable by cultivating a cell as described above.

In a further aspect, the present invention also relates to a method of producing a chimeric MAD7-type nuclease, or a sequence encoding the same, the method comprising the following steps:
(a) defining the domain structure of a MAD7 nuclease and of at least one further CRISPR nuclease, wherein the different nucleases each have a defined PAM specificity and/or overall functionality;
(b) exchanging one defined domain from the MAD7 nuclease as recipient against one domain from the at least one further CRISPR nuclease as donor and thus creating a chimeric MAD7-type nuclease; and
(c) obtaining a chimeric MAD7-type nuclease;
(d) optionally: characterizing the chimeric MAD7-type nuclease of step (c).

In one embodiment of the method of producing a chimeric MAD7-type nuclease, or a sequence encoding the same, the chimeric nuclease comprises at least one donor domain from a CRISPR class II nuclease, preferably from a CRISPR class II type V nuclease, more preferably from a CRISPR Cpf1/Cas12a nuclease.

In another aspect, the present invention also relates to a method of producing a chimeric nuclease, or a sequence encoding the same, the method comprising the following steps:
(a) defining the domain structure of a MAD7-type nuclease according to any of the embodiments described above, and of at least one further CRISPR nuclease, wherein the different nucleases each have a defined PAM specificity and/or overall functionality;
(b) exchanging one defined domain from the at least one further CRISPR nuclease as recipient against one domain from the MAD7-type nuclease as donor and thus creating a chimeric MAD7-type nuclease; and
(c) obtaining a chimeric nuclease;
(d) optionally: characterizing the chimeric nuclease of step (c).

In yet another aspect, the present invention provides a method of treating a disease in a subject, the method comprising the following steps:
(a) defining at least one mutation in the genome of a subject to be treated causing a disease:
(b) designing at least one guide nucleic acid sequence as defined above and optionally at least one repair template to modify the at least one mutation in a targeted way;
(c) introducing the MAD7-type nuclease as described in any of the embodiments above or the genome engineering system as described in any of the embodiments above or the expression construct as described in any of the embodiments above into at least one cell of a subject to be treated; and
(d) obtaining at least one cell comprising a targeted modification at the site of the at least one mutation causing a disease.

In another aspect, the present invention relates to a MAD7-type nuclease as described in any of the embodiments above, or the genome engineering system as defined in any of the embodiments described above or the expression construct as defined in any of the embodiments described above for use in a method of treating a disease in a subject.

In a further aspect, the present invention also relates to a use of a MAD7-type nuclease as described in any of the embodiments above, or the genome engineering system as defined in any of the embodiments described above or the expression construct as defined in any of the embodiments described above for modifying a genomic target site of interest, ex vivo or in vitro.

### Definitions

A “nucleic acid guided nuclease or NGN” is a site-specific nuclease, which requires a nucleic acid molecule, in particular a guide RNA, to recognize and cleave a specific target site, e.g. in genomic DNA. The nucleic acid guided nuclease forms a nuclease complex together with the guide nucleic acid and then recognizes and cleaves the target site in a sequence-dependent matter. Nucleic acid guided nucleases can therefore be programmed to target a specific site by the design of the guide nucleic acid sequence.

A “MAD7-type nuclease” is a nuclease, which is derived from a MAD7 nuclease. The MAD7-type nuclease has been altered so that it differs from the MAD7 nuclease, but it still has the same basic architecture and functionalities as the MAD7 nuclease. The MAD7 nuclease may have an amino acid sequence according to SEQ ID NO: 3 and the MAD7-type nuclease derived from it may have an amino acid sequence, which differs in certain amino acid positions from SEQ ID NO: 3. More specifically, the MAD7-type nuclease may carry mutations of single amino acids in the amino acid sequence compared to the MAD7 nuclease it is derived from. These mutations alter the PAM specificity of the nuclease to broaden or change the target range. Specific mutations providing this effect are described herein. Besides the specifically defined mutations, the MAD7-type nuclease may further differ from the MAD7 nuclease it is derived from, in particular the MAD7 nuclease having an amino acid sequence according to SEQ ID NO: 3, as long as it maintains its nuclease activity on a target region.

A nucleic acid guided nuclease recognizes a certain protospacer adjacent motif (PAM) at the target site, which is required to be present for the nuclease to cut the target site. The “PAM specificity” of a nuclease defines, which PAM(s) the nuclease recognizes. For example, certain variations of a PAM or different PAMs may result in cleavage. The different variants or different PAMs may provide a varying degree of nuclease activity at a target site. In the context of the present invention, a nuclease is considered to “recognize” a certain PAM, when the Indel percentage at a certain site with the PAM, normalized by transformation efficiency in the system used, is at least 10%, preferably at least 20%, at least 30%, at least 40%, at least 50% or at least 60%. In this context, a “higher activity” of a nuclease refers to a higher indel percentage at a certain target site compared to another nuclease.

An “indel” refers to an insertion or a deletion of one or more nucleotides at the target site, which is due to site specific nuclease activity at the target site. The frequency with which indels occur at the target site can be used as a measure for site specific nuclease activity.

A “domain” of the nuclease refers to a functional subunit of the enzyme that can be stable and folded independently. A domain is usually conserved in terms of protein sequence and tertiary structure indicating its functionality. Defining the “domain structure” of an enzyme includes identifying the functional domains of an enzyme in terms of their amino acid sequence and on a structural level.

A nucleic acid sequence is “codon optimized” when the sequence is adapted to the preferred codon usage in the organism that it is to be expressed in, i.e. a “target cell of interest”. If a nucleic acid sequence is expressed in a heterologous system, codon optimization increases the translation efficiency significantly.

A “nickase” is a nuclease, which introduces a single-strand break instead of a double-strand break. Nucleic acid guided nucleases can be rendered into nickases by introduction of certain mutations. Alternatively, they can be rendered into a “nuclease-dead variant”, which does still recognize the target sequence but is unable to cleave it.

A “nuclear localization signal” or a “nuclear localization sequence” refers to an amino acid sequence, which is added at the C-terminus and/or the N-terminus of a polypeptide or protein, which causes the polypeptide or protein to be imported into the cellular nucleus by nuclear transport.

A “genome engineering system” comprises at least one nucleic acid guided nuclease and at least one guide nucleic acid sequence, which recognizes a target sequence to be cut by the nuclease. The at least one “guide nucleic acid sequence” comprises a “scaffold region” and a “target region”. The “scaffold region” is a sequence, to which the nucleic acid guided nuclease binds to form a targetable nuclease complex. The scaffold region may comprise direct repeats, which are recognized and processed by the nucleic acid guided nuclease to provide mature crRNA. The “target region” defines the complementarity to the target site, which is intended to be cleaved.

A “genomic target region” is a region in the genome of the target cell, which is to be modified using the genome engineering system of the present invention. The target region can be an endogenous sequence, e.g. an endogenous target gene, or an isolated nucleic acid region, which is not part of the genome of the target cell but e.g. present on a plasmid or an artificial chromosome.

A “repair template” represents a single-stranded or double-stranded nucleic acid sequence, which can be provided during any genome editing causing a double-strand or single-strand DNA break to assist the targeted repair of said DNA break by providing a RT as template of known sequence assisting homology-directed repair. The RT may comprise “symmetric or asymmetric homology arms”, which provide homology to the sequences flanking the double-strand break introduced by the nuclease and thus promote error-free homology directed repair. The repair template may also comprise at least one chemically modified base or backbone.

A “chemically modified base” is present in the repair template, when at least one nucleobase has been modified to carry one or more substituent(s) or label(s) or one or more nucleotide(s) carry a molecule other than a nucleobase instead of a nucleobase. A “chemically modified back bone” is present in the repair template, when the phosphate back bone carries at least one modification such as e.g. a phosphorothioate bond.

A “self-cleaving ribozyme” is an RNA molecule that is capable to catalyze its own cleavage at a specific site. Upon transcription, self-cleaving ribozymes fold into a specific structure, sometimes requiring the presence of certain metal cations, which induces cleavage of the phosphodiester backbone at a certain position. A number of ribozymes are known, which can be used in a variety of settings.

“Suitable reagents”, which are present in the kit according to the invention for each of the compartments include any compounds and buffers, which stabilizes the respective components and ensure their activity and/or correct folding. In particular, the suitable agents may be buffers, co-factors and stabilizers.

A “targeted modification” of at least one genomic target sequence in the context of the present invention refers to any change of a (nucleic acid) sequence that results in at least one difference in the (nucleic acid) sequence distinguishing it from the original sequence. In particular, a modification can be achieved by insertion or addition of one or more nucleotide(s), or substitution or deletion of one or more nucleotide(s) of the original sequence or any combination of these. A targeted modification is introduced using site-specific tools such as a nucleic acid guided nuclease, which recognizes and cut the target at a specific location. If two or more different guide nucleic acid sequences are used, it is possible to target multiple sites in the genomic target region and introduce “multiple modifications”.

A “chimeric” nuclease comprises parts originating from different nucleases. In particular, a chimeric nuclease comprises domains from at least two different nucleases. Preferably, domains with the same function are swapped to obtain a chimeric nuclease. Thus, the nuclease maintains its functionality but can have an altered specificity or and increased activity. In particular, a chimeric MAD7-type nuclease is derived from a MAD7 nuclease by swapping one domain of the MAD7 nuclease with a corresponding domain from another CRISPR nuclease, wherein the swapped domain provides the resulting chimeric MAD7-type nuclease with an altered or broadened PAM specificity as explained herein.

A “CRISPR nuclease”, as used herein, is any nucleic acid guided nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. CRISPR nucleases also comprise mutants or catalytically active fragments or fusions of a naturally occurring CRISPR effector sequences, or the respective sequences encoding the same. A CRISPR nuclease may in particular also refer to a CRISPR nickase or even a nuclease-dead variant of a CRISPR polypeptide having endonucleolytic function in its natural environment. The CRISPR nucleases include CRISPR/Cas systems, including CRISPR/Cas9 systems, CRISPR/Cpf1 systems, CRISPR/C2C2 systems, CRISPR/CasX systems, CRISPR/CasY systems, CRISPR/Cmr systems, CRISPR/MAD7 systems, CRISPR/CasZ systems and/or any combination, variant, or catalytically active fragment thereof.

The terms “plant” or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, animal cells, fungal cells or plant cells, can have any degree of ploidity, i.e. they may either be haploid, diploid, tetraploid, hexaploid or polyploid.

A “mutation in the genome of a subject to be treated causing a disease” is an insertion, deletion or replacement of one or more nucleotides, which alters a genomic sequence of the subject with respect to the sequence in a healthy subject and thus causes a disease. For example, a single point mutation in a genomic sequence may render the expression product non-functional or significantly reduce its functionality and thus result in a disease.

Whenever the present disclosure relates to the percentage of identity of nucleic acid or amino acid sequences to each other these values define those values as obtained by using the EMBOSS Water Pairwise Sequence Alignments (nucleotide) programme (www.ebi.ac.uk/Tools/psa/emboss_water/nucleotide.html) nucleic acids or the EMBOSS Water Pairwise Sequence Alignments (protein) programme (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see www.ebi.ac.uk/Tools/psa/ and Smith, T.F. & Waterman, M.S. “Identification of common molecular subsequences” Journal of Molecular Biology, 1981 147 (1):195-197). When conducting an alignment, the default parameters defined by the EMBL-EBI are used. Those parameters are (i) for amino acid 25 sequences: Matrix = BLOSUM62, gap open penalty = 10 and gap extend penalty = 0.5 or (ii) for nucleic acid sequences: Matrix = DNAfull, gap open penalty = 10 and gap extend penalty = 0.5. The skilled person is well aware of the fact that, for example, a sequence encoding a protein can be “codon optimized” if the respective sequence is to be used in another organism in comparison to the original organism a molecule originates from.

### BRIEF DESCRIPTION OF FIGURES

FIG. 1 shows MAD7 activity on TTTN Pam sites in corn protoplasts. Both codon optimized versions of MAD7 (Version A and Version B) show similar or greater activity than the original Cpf1 (LbCpf1). The target 5 carries a TTTA PAM, the target 7 carries a TTTG PAM and the target 51 carries a TTTC PAM. Activity of the nucleases is measured in indel percentage normalized by protoplast transformation efficiency.

FIG. 2 shows the use of MAD7 with CTTN PAMs in corn protoplasts. The individual PAM for each target site is given at the bottom of the chart. Activity of the nuclease is measured in indel percentage normalized by protoplast transformation efficiency.

FIGS. 3 A shows an alignment of LbCpf1-RR version (SEQ ID NO: 73) vs. MAD7 (SEQ ID NO: 74) to find common motifs for making a MAD7-RR version. The consensus sequence as determined by this direct comparison is shown in the middle. With reference to MAD7 (codonoptimzed; SEQ ID NO: 3): D537=GAC (nucleotide positions 1609-1611) converts to R537=CGT and K602=AAG (nucleotide positions 1804-1806) converts to R602=AGG. B shows an alignment of LbCpf1-RVR version (SEQ ID NO: 75) vs MAD7 (SEQ ID NO: 76) to find common motifs for making MAD7 RVR version. The consensus sequence as determined by this direct comparison is shown in the middle. With reference to MAD7 (codon-optimized; SEQ ID NO: 3): D537=GAC (nucleotide positions 1609-1611) converts to R537=CGT, K543=AAG (nucleotide positions 1627-1629) converts to V543=GTA and N547=AAC (nucleotide positions 1639-1641) converts to R547=AAG.

FIG. 4 shows a comparison of MAD7-RR and LbCpf1-RR activity on TYCV PAM sites in corn protoplasts. MAD7-RR demonstrates activity on TYCV sites but not as good as LbCpf1-RR. The target 77 carries a TTCA PAM, the target 82 carries a TCCC PAM. Activity of the nucleases is measured in indel percentage normalized by protoplast transformation efficiency.

FIG. 5 shows a comparison of the activity of MAD7 and MAD7-V1 at four CTTN PAM sites. MAD7-V1 shows similar or higher activity than MAD7 in three out of four targets tested. The target 14 carries a CTTC PAM and the targets 15, 20 and 43 carry a CTTG PAM. Activity of the nucleases is measured in indel percentage normalized by protoplast transformation efficiency.

FIG. 6 shows a comparison of the activity of MAD7-RR and MAD7-RRR at TYCV PAM sites. MAD7-RRR shows two times higher activity than MAD7-RR. crGEP77 carries a TTCA PAM and crGEP82 carries a TCCC PAM. Activity of the nucleases is measured in indel percentage normalized by protoplast transformation efficiency.

FIG. 7 shows a schematic of two guide RNA expression strategies in the multiplexing editing experiments. Guide RNA expression as individual guide RNA is shown in FIG. 7A. m7GEP1 is used as an example. Guide RNA array is exemplified in FIG. 7B. Scaffold corresponds to SEQ ID NO: 5 and DR represents a partial scaffold sequence corresponding to SEQ ID NO: 49.

FIG. 8 shows the results of multiplex editing in corn with MAD7-V1. FIGS. 8A and 8C show the editing results using a mixture of five individual guide RNAs targeting five different target sides. FIGS. 8B and 8D are targeting the same sites as in FIGS. 8A and 8C respectively but using guide RNA arrays. FIGS. 8E and 8F show editing results targeting two pairs of target sites in two genes using mixture of individual guide RNAs (8E) or with guide RNA array (8F).

FIG. 9 shows the results of testing sequence-optimized MAD7 in wheat embryos. In FIG. 9A, the editing efficiency for the three tested wheat genomes is shown as a diagram. FIG. 9B additionally lists the PAMs and the target sequences (SEQ ID NOs: 58 to 72).

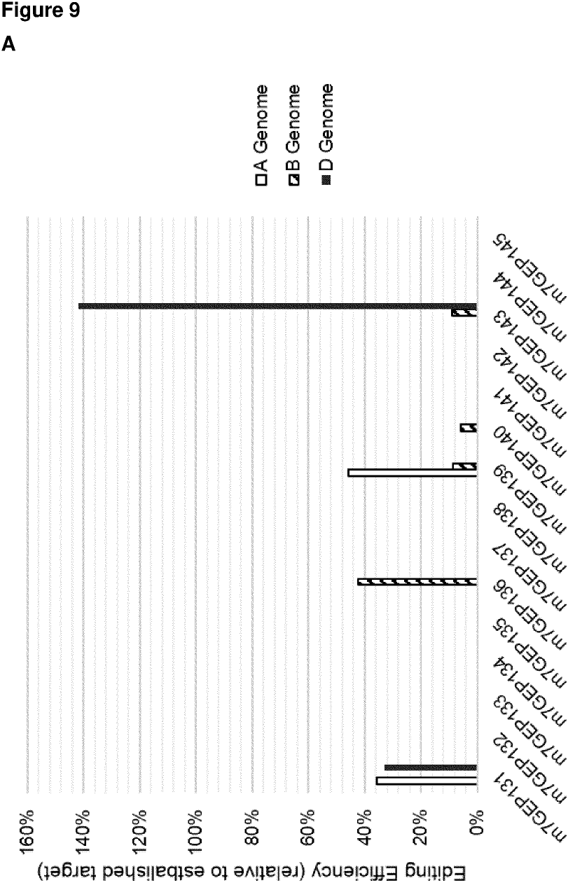

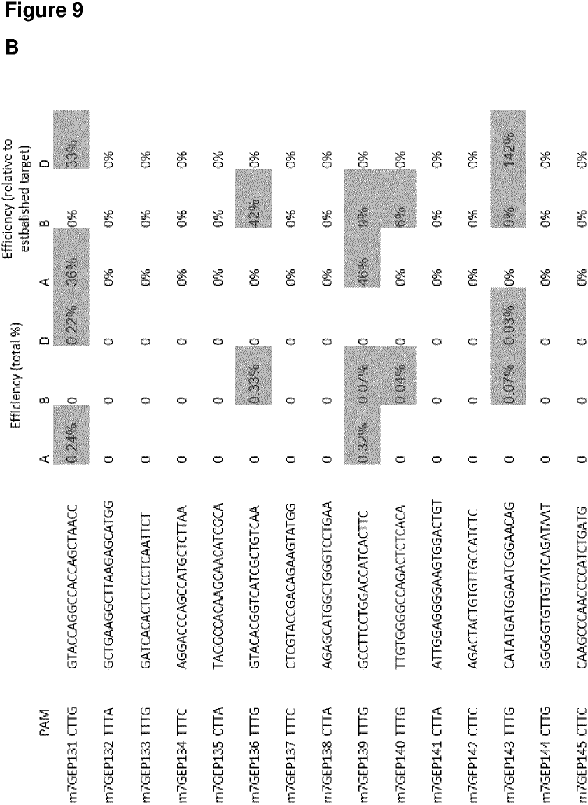

Sequences:

| | |
|---|---|
| SEQ ID NO: 1 | cDNA of codon-optimized MAD7 version A |
| SEQ ID NO: 2 | cDNA of codon-opimized MAD7 version B |
| SEQ ID NO: 3 | MAD protein encoded by codon-optimized MAD7 versions A and B |
| SEQ ID NO: 4 | BdUbi10 promoter (Brachypodium distachyon) |
| SEQ ID NO: 5 | 35 bp MAD7 scaffold sequence |
| SEQ ID NO: 6 | Hammerhead ribozyme |
| SEQ ID NO: 7 | Hepatitis-delta virus (HDV) ribozyme |
| SEQ ID NO: 8 | ZmUbi1 promoter |
| SEQ ID NO: 9 | ZmUbi1 intron |
| SEQ ID NO: 10 | cDNA of codon-optimized MAD7 RR version A |
| SEQ ID NO: 11 | cDNA of codon-optimized MAD7 RR version B |
| SEQ ID NO: 12 | Protein MAD7 RR encoded by cDNA of condon-optimized MAD7 RR versions A and B |
| SEQ ID NO: 13 | cDNA of codon-optimized MAD7 RVR version A |
| SEQ ID NO: 14 | cDNA of codon-optimized MAD7 RVR version B |
| SEQ ID NO: 15 | Protein MAD7 RVR encoded by cDNA of codon-optimized MAD7 RVR versions A and B |
| SEQ ID NO: 16 | cDNA of codon-optimized mutated MAD7-V1 version A |
| SEQ ID NO: 17 | cDNA of codon-optimized mutated MAD7-V1 version B |
| SEQ ID NO: 18 | Protein MAD7-V1 encoded by cDNA of codon-optimized MAD7-V1 versions A and B |
| SEQ ID NO: 19 | cDNA of codon-optimized mutated MAD7-V2 version A |
| SEQ ID NO: 20 | cDNA of codon-optimized mutated MAD7-V2 version B |
| SEQ ID NO: 21 | Protein MAD7-V2 encoded by cDNA of codon-optimized MAD7-V2 versions A and B |
| SEQ ID NO: 22 | cDNA of codon-optimized MAD7 RRR version A |
| SEQ ID NO: 23 | cDNA of codon-optimized MAD7 RRR version B |
| SEQ ID NO: 24 | Protein MAD7 RRR encoded by cDNA of codon-optimized MAD7 RRR versions A and B |
| SEQ ID NO: 25 | cDNA of codon-optimized MAD7 RRVR version A |
| SEQ ID NO: 26 | cDNA of codon-optimized MAD7 RRVR version B |
| SEQ ID NO: 27 | Protein MAD7 RRVR encoded by cDNA of codon-optimized MAD7 RRVR versions A and B |
| SEQ ID NO: 28 | cDNA of codon-optimized mutated MAD7-V1 version A including additional N272A mutation |
| SEQ ID NO: 29 | cDNA of codon-optimized mutated MAD7-V1 version B including additional N272A mutation |
| SEQ ID NO: 30 | Protein MAD7-V1 + N272A encoded by cDNA of codon-optimized MAD7-V1 + N272A versions A and B |
| SEQ ID NO: 31 | cDNA of codon-optimized mutated MAD7-V2 version A including additional N272A mutation |
| SEQ ID NO: 32 | cDNA of codon-optimized mutated MAD7-V2 version B including additional N272A mutation |
| SEQ ID NO: 33 | Protein MAD7-V2 + N272A encoded by cDNA of codon-optimized MAD7-V2 + N272A versions A and B |
| SEQ ID NO: 34 | cDNA of codon-optimized MAD7 RRR version A including additional N272A mutation |
| SEQ ID NO: 35 | cDNA of codon-optimized MAD7 RRR version B including additional N272A mutation |
| SEQ ID NO: 36 | Protein MAD7 RRR + N272A encoded by cDNA of codon-optimized MAD7 RRR + N272A versions A and B |
| SEQ ID NO: 37 | cDNA of codon-optimized MAD7 RRVR version A including additional N272A mutation |
| SEQ ID NO: 38 | cDNA of codon-optimized MAD7 RRVR version B including additional N272A mutation |
| SEQ ID NO: 39 | Protein MAD7 RRVR + N272A encoded by cDNA of codon-optimized MAD7 RRVR + N272A versions A and B |
| SEQ ID NO: 40 | MAD7-Cpf1 chimera I |
| SEQ ID NO: 41 | MAD7-Cpf1 chimera II |
| SEQ ID NO: 42 | Mad7 |
| SEQ ID NO: 43 | LbCpf1 |
| SEQ ID NO: 44 | LbCpf1 RR |
| SEQ ID NO: 45 | LbCpf1 RVR |
| SEQ ID NO: 46 | AsCpf1 |
| SEQ ID NO: 47 | As Cpf1 RR |
| SEQ ID NO: 48 | AsCpf1 RVR |
| SEQ ID NO: 49 | partial MAD7 scaffold sequence (AATTTCTACTCTTGTAGAT) |
| SEQ ID NO: 50 | HMG13 guide RNA sequence 1 from Example 11, Table 6 |
| SEQ ID NO: 51 | HMG13 guide RNA sequence 2 from Example 11, Table 6 |
| SEQ ID NO: 52 | ZmCPL3 guide RNA sequence 1 from Example 11, Table 6 |
| SEQ ID NO: 53 | ZmCPL3 guide RNA sequence 2 from Example 11, Table 6 |
| SEQ ID NO: 54 | ZmCPL1 guide RNA sequence 1 from Example 11, Table 6 |
| SEQ ID NO: 55 | ZmCPL1 guide RNA sequence 2 from Example 11, Table 6 |
| SEQ ID NO: 56 | regeneration booster protein 2 (RBP2) |
| SEQ ID NO: 57 | regeneration booster protein 2 (coding sequence) |
| SEQ ID NO: 58 | target sequence 1 from FIG. 9B |
| SEQ ID NO: 59 | target sequence 2 from FIG. 9B |
| SEQ ID NO: 60 | target sequence 3 from FIG. 9B |
| SEQ ID NO: 61 | target sequence 4 from FIG. 9B |
| SEQ ID NO: 62 | target sequence 5 from FIG. 9B |
| SEQ ID NO: 63 | target sequence 6 from FIG. 9B |
| SEQ ID NO: 64 | target sequence 7 from FIG. 9B |
| SEQ ID NO: 65 | target sequence 8 from FIG. 9B |
| SEQ ID NO: 66 | target sequence 9 from FIG. 9B |
| SEQ ID NO: 67 | target sequence 10 from FIG. 9B |
| SEQ ID NO: 68 | target sequence 11 from FIG. 9B |
| SEQ ID NO: 69 | target sequence 12 from FIG. 9B |
| SEQ ID NO: 70 | target sequence 13 from FIG. 9B |
| SEQ ID NO: 71 | target sequence 14 from FIG. 9B |
| SEQ ID NO: 72 | target sequence 15 from FIG. 9B |

### DETAILED DESCRIPTION

The present invention establishes activity in plants of a previously uncharacterized nuclease and expands the recognition of PAM sites by the MAD7 nuclease from YTTN to TYCV, TATV and TTCN. The invention provides codon optimized versions of MAD7 and shows that the MAD7 scaffold sequence in corn protoplasts leads to the formation of indels indicating activity at target sites using plant gene expression elements. It is demonstrated that by certain amino acid substitution, the PAM recognition can be expanded to cover a wider target range with sufficient activity for genome editing. This provides a specific advantage for developing genome scale editing capabilities.

The present invention provides a nucleic acid guided nuclease, wherein the nuclease is a MAD7-type nuclease, or a sequence encoding the same, with an engineered PAM specificity, wherein the nuclease is engineered to recognize at least one PAM selected from the group consisting of TYCV, TATV, or TTCN.

MAD7 nuclease is a freely distributed nuclease from Inscripta Genomics company, which shows its highest activity with YTTN (i.e., CTTN or TTTN) PAMs, while with other PAMs, the nuclease is significantly less active and therefore not suitable for efficient application. In order to expand the target range, the present invention provides MAD7-type nucleases, which differs from a MAD7 nuclease in that it is engineered to (additionally) recognize TYCV, TATV and/or TTCN PAM(s).

The MAD7-type nuclease according to the invention preferably shows an indel percentage at a certain site with a TYCV, TATV and/or TTCN PAM of at least 10%, preferably at least 20%, at least 30%, at least 40%, at least 50% or at least 60%, wherein the indel percentage is normalized by transformation efficiency in the system used.

A PAM is generally considered workable, when, if five different guides are tested, at least one has over 10%, preferably over 20% indel percentage normalized by transformation efficiency in the system used. At least one out of 5 is what was observed with LbCpf1 on TTTV PAMs. With MAD7 working with TTTN PAMs, 4 out of 7 and 3 out of 5 were observed in two cases. When MAD7 was tested with CTTN PAMs, the frequency was low (<1 out of 20), although one site was found that showed >20% Indel percentage.

In one embodiment of the nucleic acid guided nuclease described above, the nucleic acid guided nuclease additionally recognizes at least one PAM selected from the group consisting of TTTN and CTTN.

Advantageously, the MAD7-type nuclease of the present invention, does not only recognize previously not suitable PAMs but it also still recognizes YTTN PAMs like a MAD7 nuclease to a sufficient degree, i.e. leading to an indel percentage of at least 10%, preferably at least 20%, at least 30%, at least 40%, at least 50% or at least 60%, wherein the indel percentage is normalized by transformation efficiency in the system used. Thus, the nucleases of the present invention do not lose their ability to recognize YTTN PAMs but broaden the application range.

In one embodiment of the nucleic acid guided nuclease described above, the nucleic acid guided nuclease according to any of the embodiments above, has a higher activity on a CTTN site in comparison to a MAD7 nuclease.

The MAD7-type nuclease of the present invention can also show an even higher activity on a site carrying a CTTN PAM compared to a MAD7 nuclease. Thus, the MAD7-type nuclease may provide an improved efficiency over MAD7 in some applications.

In the context of the present invention, it was found out that certain amino acid replacements in the amino acid sequence of a MAD7 nuclease provide the desired altered or expanded PAM recognition without leading to significant losses in efficiency or specificity of the nuclease.

In one embodiment of the nucleic acid guided nuclease described above, the nucleic acid guided nuclease according to any of the embodiments above, comprises at least one mutation at position 177, 272, 537, 543, 547, or 602 in comparison to the reference sequence of the MAD7 nuclease according to SEQ ID NO:3, or a combination of mutations. More specifically, the at least one mutation is independently selected from K177R, N272A, D537R, K543V, K543R, N547R, K602R, or a combination thereof.

The amino acid positions are given with respect to the amino acid sequence of SEQ ID NO: 3, which is derived from the original MAD7 nuclease (SEQ ID NO: 42) that was used as a basis for the developments of the present invention, with the addition of nuclear localization signals (NLS). As demonstrated in the examples below, certain combinations of the above-mentioned mutations provide active nucleases with altered or expanded PAM specificity allowing a broad range of application.

In one embodiment of the nucleic acid guided nuclease described above, the nucleic acid guided nuclease according to any of the embodiments above, or a domain thereof, comprises a combination of mutations selected from D537R+K602R (MAD7-RR), D537R+K543V+N547R (MAD7-RVR), K177R+D537R (MAD7-V1), K177R+D537R+K543R (MAD7-V2), K177R+D537R+K602R (MAD7-RRR), K177R+D537R+K543V+N547R (MAD7-RRVR), K177R+D537R+N272A (MAD7-V1 + N272A), K177R+D537R+N272A+K543R (MAD7-V2 + N272A), K177R+D537R+N272A+K602R (MAD7-RRR + N272A), K177R+D537R+K543V+N272A+N547R (MAD7-RRVR + N272A).

The names of the respective mutants are given in parentheses and the amino acid positions are given with respect to the amino acid sequence of SEQ ID NO: 3. The mutants are further characterized by their full amino acid sequences and the respective nucleic acid sequences encoding the same. The nucleic acid sequences comprise two different codon optimized versions (versions A and B) for the expression in plants, in particular corn. Versions, which a codon optimized for other target systems are also covered.

In one embodiment of the nucleic acid guided nuclease described above, the nucleic acid guided nuclease according to any of the embodiments above comprises an amino acid sequence of any one of SEQ ID NOs: 12, 15, 18, 21, 24, 27, 30, 33, 36, 39, 40 or 41 or an amino acid sequence having at least 90%, 91%, 92%, 93%, 94%, 95%, 96%, 97%, 98% or 99% sequence identity to the respective amino acid sequence, or the nuclease is encoded by a nucleic acid sequence of any one of SEQ ID NOs: 10, 11, 13, 14, 16, 17, 19, 20, 22, 23, 25, 26, 28, 29, 31, 32, 34, 35, 37, 38 or a codon optimized variant thereof, or a nucleic acid sequence encoding an amino acid sequence having at least 90%, 91%, 92%, 93%, 94%, 95%, 96%, 97%, 98% or 99% sequence identity to any one of the respective SEQ ID NOs: 12, 15, 18, 21, 24, 27, 30, 33, 36, 39, 40 or 41.

The SEQ ID NOs of the mutants are assigned as shown in table 1 below.

TABLE 1

| | Codon optimized version A | Codon optimized version B | |
|---|---|---|---|
| | DNA | DNA | protein |
| MAD7 | SEQ ID NO: 1 | SEQ ID NO: 2 | SEQ ID NO: 3 |
| MAD7 RR | SEQ ID NO: 10 | SEQ ID NO: 11 | SEQ ID NO: 12 |
| MAD7 RVR | SEQ ID NO: 13 | SEQ ID NO: 14 | SEQ ID NO: 15 |
| MAD7-V1 | SEQ ID NO: 16 | SEQ ID NO: 17 | SEQ ID NO: 18 |
| MAD7-V2 | SEQ ID NO: 19 | SEQ ID NO: 20 | SEQ ID NO: 21 |
| MAD7-RRR | SEQ ID NO: 22 | SEQ ID NO: 23 | SEQ ID NO: 24 |
| MAD7-RRVR | SEQ ID NO: 25 | SEQ ID NO: 26 | SEQ ID NO: 27 |
| MAD7-V1 + N272A | SEQ ID NO: 28 | SEQ ID NO: 29 | SEQ ID NO: 30 |
| MAD7-V2 + N272A | SEQ ID NO: 31 | SEQ ID NO: 32 | SEQ ID NO: 33 |
| MAD7-RRR + N272A | SEQ ID NO: 34 | SEQ ID NO: 35 | SEQ ID NO: 36 |
| MAD7-RRVR + N272A | SEQ ID NO: 37 | SEQ ID NO: 38 | SEQ ID NO: 39 |

The skilled person is well aware of how a sequence encoding a protein is codon optimized if the respective sequence is to be used in another organism in comparison to the original organism a molecule originates from. Therefore, the skilled person can provide a codon-optimized variant of the respective nucleic acid sequences given above in order to use them in a different organism.

In one embodiment of the nucleic acid guided nuclease described above, the nucleic acid guided nuclease according to any of the embodiments above comprises at least one mutation rendering the nuclease to a nickase or to a nuclease-dead variant of the nuclease, preferably the nuclease comprises a D885A and/or a E970A mutation or the nuclease comprises a R1181A mutation in comparison to the reference sequence of the MAD7 nuclease according to SEQ ID NO:3.

For some applications, it may be desirable to use the nucleic acid guided nuclease of the present invention to target a genomic site of interest without introducing a double strand break. In such cases the MAD7-type nuclease may be altered so that it has nickase activity inducing a single strand break, or it may be turned into a nuclease-dead or nuclease-deficient variant, which does not induce any breaks at the target site.

In one embodiment of the nucleic acid guided nuclease described above, the nucleic acid guided nuclease according to any of the embodiments above comprises at least one nuclear localization signal, preferably wherein the nuclease comprises one nuclear localization signal at the N-terminus and one nuclear localization signal at the C-terminus.

In order to exert its effect on the genome of a target cell, it can be advantageous to target the MAD7-type nuclease of the present invention for import into the nucleus. To achieve import in the nucleus by nuclear transport mechanisms, the MAD7-type nuclease is modified so that it comprises a nuclear localization signal at the N-terminus and/or at the C-terminus.

In one embodiment of the nucleic acid guided nuclease described above, the nucleic acid sequence encoding the nucleic acid guided nuclease according to any of the embodiments above is codon optimized for expression in a target cell of interest.

As mentioned above, the skilled person can provide a codon-optimized variant of the nucleic acid sequence encoding the MAD7-type nuclease in order to use it in a different organism, preferably different plant or plant species.

The present invention also relates to a genome engineering system comprising at least one MAD7-type nuclease according to any of the embodiments described above, or a sequence encoding the same, and at least one guide nucleic acid sequence, or a sequence encoding the same, wherein the at least one guide nucleic acid sequence comprises a scaffold region and a targeting region.

The genome engineering system of the invention comprises the MAD7-type nuclease described above and at least one guide nucleic acid sequence. The MAD7-type nuclease may comprise any of the sequences, mutations and combinations of mutations defined above. The at least one guide nucleic acid is preferably a CRISPR RNA (crRNA) or a pre-crRNA, which is sufficient by itself and does not require the presence of a trans-activating CRISPR RNA (tra-crRNA) for targeting. The one guide nucleic acid sequence comprises scaffold region and a targeting region. The scaffold region represents the recognition and binding site for the MAD7-type nuclease to form a targetable nuclease complex, which can then induce a double strand break in a target sequence. The scaffold region may comprise a nuclease recognition site comprising direct repeats, which are recognized and processed by the nuclease to provide mature crRNA. MAD7 can not only cut DNA but it also has ribonuclease activity, which it uses to process its pre-crRNA to provide mature crRNA (Safari et al., CRISPR Cpf1 proteins: struc-ture, function and implications for genome editing, Cell & Bioscience (2019), 9:36). The scaf-fold region may advantageously be designed for MAD7 recognition and/or processing. The targeting region provides the complementary to the target sequence and thus allows the nu-clease to recognize and cleave the target site.

In one embodiment of the genome engineering system described above, the targeting region targets a genomic target region of interest, which is an endogenous or isolated nucleic acid region of a eukaryotic cell.

A genomic target region to be modified may be a coding region of a target gene or it may be a regulatory sequence. The target region may be an endogenous sequence, e.g. an endogenous target gene or it may be an isolated nucleic acid region, which is not part of the genome of the target cell but e.g. present on a plasmid or an artificial chromosome.

In one embodiment of the genome engineering system according to any of the embodiments described above, the genomic target region of interest is an endogenous or isolated nucleic acid region of a plant cell or organism.

Advantageously, the genome engineering system of the present invention can be used in a wide range of plants. Due to the expanded PAM specificity of the MAD7-type nuclease described above, it is possible to apply the system on genomes, which were previously not accessible due to a lack of suitable PAMs. The skilled person is aware of how to design suitable guide nucleic acid sequences for a certain application. In case a sequence encoding the MAD7-type nuclease is used, it may be desirable to provide a codon optimized variant of the sequence for the particular target organism, in which the system is to be used in order to achieve efficient expression of the nuclease.

In one embodiment of the genome engineering system according to any of the embodiments described above, the system additionally comprises at least one repair template, or a sequence encoding the same.

A repair template can be provided together with the MAD7-type nuclease of the present invention, so that the double-strand or single-strand DNA break caused by the nuclease is repaired by homologous recombination between the genomic target region and the repair template. Thus, it is possible to introduce a targeted modification, e.g. an insertion of a specific sequence, at the target site. The repair template may be single-stranded or double-stranded and may also comprise symmetric or asymmetric homology arms, which provide homology to the sequences flanking the break and thus promote error-free homology directed repair.

In one embodiment of the genome engineering system according to any of the embodiments described above, the at least one repair template comprises or encodes a double- and/or single-stranded sequence.

In one embodiment of the genome engineering system according to any of the embodiments described above, the at least one repair template comprises symmetric or asymmetric homology arms.

Furthermore, the repair template may comprise one or more chemically modified base(s) or backbone. When using such repair templates, it becomes possible to introduce certain modifications into the genomic target region and furnish the target region, which certain properties. For example, nucleotides may be substituted or labelled in a certain way, changing their properties or rendering them traceable. Furthermore, phosphorothioate nucleotides may be introduced for further applications.

In one embodiment of the genome engineering system according to any of the embodiments described above, the at least one MAD7-type nuclease, or the sequence encoding the same, and/or the at least one guide nucleic acid, or the sequence encoding the same, and/or optionally the at least one repair template, or the sequence encoding the same, are provided simultaneously, or one after another.

Depending on the delivery method used, the components of the genome engineering system of the present invention, may be provided to the target cell simultaneously or one after the other. The components may be provided as one or two or more different expression constructs to be introduced into the cell or they may be provided as protein and, respectively, nucleic acid constructs.

The present invention also relates to an expression construct comprising or encoding at least one MAD7-type nuclease as described in any of the embodiments above, and/or at least one guide nucleic acid sequence as defined above, and/or at least one repair template.

The expression construct may comprise regulatory sequences including promoter and terminator sequences. Furthermore, the expression construct may comprise codon optimized sequences for efficient expression in a certain organism. The MAD7-type nuclease encoded in the expression construct may comprise any of the sequences, mutations and combinations of mutations defined above.

In one embodiment, the expression construct described above comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

Suitable promoters are available to the skilled person and may be chosen depending on the setting, in which the expression construct according to the invention is used. Furthermore, the expression construct may comprise an intron, which may enhance the expression of the expression construct according to the invention.

In one embodiment of the expression construct according to any of the embodiments described above, the the regulatory sequence comprises or encodes at least one promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In another embodiment of the expression construct according to any of the embodiments described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

Certain combinations of promoters and introns are particularly preferred as they enhance the expression of the construct and can thus increase the efficiency of the system.

In one embodiment of the expression construct according to any of the embodiments described above, the construct comprises or encodes a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

The expression construct may also comprise at least one ribozyme, which upon transcription cleaves the transcript at one or more predetermined location(s). If placed strategically, the ribozyme(s) release and activate the components of the expression construct from the transcript. For example, the sequence encoding the MAD7-type nuclease may be flanked by a ribozyme at the 5′- and at the 3′-end. The guide nucleic acid sequence(s) may be included between ribozyme and nuclease sequence and can be processed by the MAD7-type nuclease itself to provide mature crRNA.

In one embodiment of the expression construct according to any of the embodiments described above, the construct comprises or encodes at least one self-cleaving ribozyme, preferably at least one hammerhead ribozyme and/or a hepatitis-delta virus (HDV) ribozyme.

In addition, the expression construct may comprise at least one terminator, which mediates transcriptional termination at the end of the expression construct or the components thereof and release of the transcript from the transcriptional complex.

In one embodiment of the expression construct according to any of the embodiments described above, the regulatory sequence comprises or encodes at least one terminator selected from the group consisting of nosT, a double 35S terminator, a ZmEf1 terminator, an AtSac66 terminator, an octopine synthase (ocs) terminator, or a pAG7 terminator, or a combination thereof.

The present invention also provides a kit comprising, in separate form, at least one compartment comprising at least one MAD7-type nuclease as described in any of the embodiments above, or a sequence encoding the same, and optionally at least one guide nucleic acid sequence as defined above, or a sequence encoding the same, and optionally at least one repair template, or a sequence encoding the same, wherein the kit additionally comprises suitable reagents for each of the at least one compartment.

Suitable reagents present in the kit according to the invention for each of the compartments include compounds and buffers, which stabilizes the respective components and ensure their activity and/or correct folding. In particular, the suitable agents are buffers, co-factors and stabilizers.

The MAD7-type nuclease of the present invention can be used in genome editing approaches. By introducing the nuclease and a guide nucleic acid sequence or a genome engineering system or an expression construct as described above into a cell, a target gene or regulatory sequence can be precisely modified. Due to the expanded PAM specificity, a large range of organisms can be targeted.

The present invention also relates to a method for the targeted modification of at least one genomic target sequence in a cell, wherein the method comprises the following steps:
(a) introducing into the cell
   (i) at least one MAD7-type nuclease, or a sequence encoding the same, as described in any of the embodiments above, and at least one guide nucleic acid sequence, or a sequence encoding the same, as defined above; or
   (ii) at least one genome engineering system as defined above or at least one expression construct as defined above,
   (iii) and, optionally at least one repair template, or a sequence encoding the same;
(b) cultivating the cell under conditions allowing the expression and/or assembly of the genome engineering system comprising the at least one MAD7-type nuclease and the at least one guide nucleic acid sequence and optionally the at least one repair template; and
(c) obtaining at least one modified cell.

Preferably, the sequence encoding the MAD7-type nuclease is codon optimized for expression in the cell, into which it is introduced.

Introducing the MAD7-type nuclease or the sequence encoding the same and the at least one guide nucleic acid sequence or the genome engineering system and optionally the repair tem-plate in step (a) may be achieved by biological or physical means, including transfection, trans-formation, including transformation by Agrobacterium spp., preferably by Agrobacterium tume-faciens, a viral vector, biolistic bombardment, transfection using chemical agents, including polyethylene glycol transfection, or any combination thereof.

Any suitable delivery method to introduce the components (i) or (ii) and optionally (iii) into a cell can be applied, depending on the target cell. The term “introducing” as used herein thus implies a functional transport of a biomolecule or genetic construct (DNA, RNA, single- or dou-ble-stranded, protein, comprising natural and/or synthetic components, or a mixture thereof) into a cell or into a cellular compartment of interest, e.g. the nucleus or an organelle, or into the cytoplasm, which allows the transcription and/or translation and/or the catalytic activity and/or binding activity, including the binding of a nucleic acid molecule to another nucleic acid molecule, including DNA or RNA, or the binding of a protein to a target structure within the cell, and/or the catalytic activity of an enzyme such introduced, optionally after transcription and/or translation.

A variety of delivery techniques may be suitable according to the methods of the present in-vention for introducing the components (i) or (ii) and optionally (iii) into a cell, in particular a plant cell, the delivery methods being known to the skilled person, e.g. by choosing direct delivery techniques ranging from polyethylene glycol (PEG) treatment of protoplasts, proce-dures like electroporation, microinjection, silicon carbide fiber whisker technology, viral vector mediated approaches and particle bombardment. A common biological means is transfor-mation with Agrobacterium spp. which has been used for decades for a variety of different plant materials. Viral vector mediated plant transformation represents a further strategy for introducing genetic material into a cell of interest.

In step (b), the MAD7-type nuclease is expressed and recognizes, and optionally processes, the guide nucleic acid sequence(s) to form a targetable nuclease complex. The guide nucleic acid sequence(s) is/are designed to target one or more predetermined genomic target regions. According to the available PAMs in the genomic target region, the nuclease and guide nucleic acid sequence(s) can be chosen or designed following to the teaching of the present invention. The nuclease introduces a single or double strand break in the target region, which is then repaired resulting in a modification, usually an insertion or a deletion. If a repair template is introduced as well, the break is repaired by homology directed repair providing a precise editing outcome.

In one embodiment of the method for targeted modification of at least one genomic target sequence in a cell, the at least one MAD7-type nuclease or sequence encoding the same, has an engineered PAM specificity, wherein the nuclease is engineered to recognize at least one PAM selected from the group consisting of TYCV, TATV, or TTCN.

In one embodiment of the method for targeted modification of at least one genomic target sequence in a cell according to any of the embodiments described above, the nuclease additionally recognizes at least one PAM selected from the group consisting of TTTN and CTTN.

In one embodiment of the method for targeted modification of at least one genomic target sequence in a cell according to any of the embodiments described above, the nuclease has a higher activity on a CTTN site in comparison to a MAD7 nuclease.

In one embodiment of the method for targeted modification of at least one genomic target sequence in a cell according to any of the embodiments described above, the nuclease, or a domain thereof, comprises at least one mutation at position 177, 272, 537, 543, 547, or 602 in comparison to the reference sequence of the MAD7 nuclease according to SEQ ID NO:3, or a combination of mutations.

In one embodiment of the method for targeted modification of at least one genomic target sequence in a cell according to any of the embodiments described above, the at least one mutation is independently selected from K177R, N272A, D537R, K543V, K543R, N547R, K602R, or a combination thereof.

In one embodiment of the method for targeted modification of at least one genomic target sequence in a cell according to any of the embodiments described above, the nuclease, or a domain thereof, comprises a combination of mutations selected from D537R+K602R (MAD7-RR), D537R+K543V+N547R (MAD7-RVR), K177R+D537R (MAD7-V1), K177R+D537R+K543R (MAD7-V2), K177R+D537R+K602R (MAD7-RRR), K177R+D537R+K543V+N547R (MAD7-RRVR), K177R+D537R+N272A (MAD7-V1 + N272A), K177R+D537R+N272A+K543R (MAD7-V2 + N272A), K177R+D537R+N272A+K602R (MAD7-RRR + N272A), K177R+D537R+K543V+N272A+N547R (MAD7-RRVR + N272A).

In one embodiment of the method for targeted modification of at least one genomic target sequence in a cell according to any of the embodiments described above, the nuclease comprises an amino acid sequence of any one of SEQ ID NOs: 12, 15, 18, 21, 24, 27, 30, 33, 36, 39, 40 or 41 or an amino acid sequence having at least 90%, 91%, 92%, 93%, 94%, 95%, 96%, 97%, 98% or 99% sequence identity to the respective amino acid sequence, or the nuclease is encoded by a nucleic acid sequence of any one of SEQ ID NOs: 10, 11, 13, 14, 16, 17, 19, 20, 22, 23, 25, 26, 28, 29, 31, 32, 34, 35, 37, 38 or a codon optimized variant thereof, or a nucleic acid sequence encoding an amino acid sequence having at least 90%, 91%, 92%, 93%, 94%, 95%, 96%, 97%, 98% or 99% sequence identity to any one of the respective SEQ ID NOs: 12, 15, 18, 21, 24, 27, 30, 33, 36, 39, 40 or 41.

Furthermore, the nuclease may comprise at least one mutation rendering it to a nickase or a nuclease-dead variant as described above. Also, the nuclease may comprise at least one nuclear localization signal, preferably the nuclease comprises one nuclear localization signal at the N-terminus and one nuclear localization signal at the C-terminus.

In one embodiment of the method for targeted modification of at least one genomic target sequence in a cell according to any of the embodiments described above, the at least one repair template comprises or encodes a double- and/or single-stranded sequence and/or the at least one repair template comprises symmetric or asymmetric homology arms and/or the at least one repair template comprises at least one chemically modified base and/or backbone.

In one embodiment of the method for targeted modification of at least one genomic target sequence in a cell according to any of the embodiments described above, the expression construct comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

In one embodiment of the method for targeted modification of at least one genomic target sequence in a cell according to the embodiment described above, the regulatory sequence comprises or encodes at least one promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In one embodiment of the method for targeted modification of at least one genomic target sequence in a cell according to the embodiment described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In one embodiment of the method for targeted modification of at least one genomic target sequence in a cell according to any of the embodiments described above, the expression construct comprises or encodes a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In one embodiment of the method for targeted modification of at least one genomic target sequence in a cell according to any of the embodiments described above, the expression construct comprises or encodes at least one self-cleaving ribozyme, preferably at least one hammerhead ribozyme and/or a hepatitis-delta virus (HDV) ribozyme).

In one embodiment of the method for targeted modification of at least one genomic target sequence in a cell according to the embodiment described above, the regulatory sequence comprises or encodes at least one terminator selected from the group consisting of nosT, a double 35S terminator, a ZmEf1 terminator, an AtSac66 terminator, an octopine synthase (ocs) terminator, or a pAG7 terminator, or a combination thereof.

Depending on the delivery method used, it is possible to introduce components (i) or (ii) and optionally (iii) as one expression construct or as part of one transformation vector so that they are delivered simultaneously into the cell. Alternatively, they can be introduced one after the other. Moreover, the components may be transiently introduced into the cell so that they are only temporarily expressed and afterwards degraded by the cell, or they may be stably introduced and expressed, e.g. by integration into the genome of the cell.

In one embodiment of the method for targeted modification of at least one genomic target sequence in a cell according to any of the embodiments described above, (i), (ii), and optionally (iii) is/are introduced simultaneously or one after another.

In one embodiment of the method for targeted modification of at least one genomic target sequence in a cell according to any of the embodiments described above, at least one of (i), (ii), and optionally (iii) is/are transiently introduced into and/or expressed in the cell.

In one embodiment of the method for targeted modification of at least one genomic target sequence in a cell according to any of the embodiments described above, at least one of (i), (ii), and optionally (iii) is/are stably introduced into and/or expressed in the cell.

Using the method described above, it is also possible to simultaneously introduce multiple modifications within the genome of the target cell by delivering multiple guide nucleic acid sequences, which target different locations in the genome of the target cell.

In one embodiment of the method for targeted modification of at least one genomic target sequence in a cell according to any of the embodiments described above, at least two, three, four, five or more different guide nucleic acid sequences, or sequences encoding the same, are introduced into the cell to make multiple modifications in the cell simultaneously.

Different kinds of modifications can be introduced into the genome of the target cell depending on the desired outcome. For example, one or more nucleotides can be inserted or deleted or exchanged. Moreover, a wide range of cells and organisms can be targeted as, due to the expanded PAM specificity, a suitable nuclease and guide nucleic acid sequence(s) combination can be chosen for any organisms of interest. Furthermore, the sequence introduced into the target cell, which encodes the MAD7-type nuclease, can be codon optimized for expression in the target organism to optimize efficiency.

In one embodiment of the method for targeted modification of at least one genomic target sequence in a cell according to any of the embodiments described above, the targeted modification of the at least one genomic target sequence in a cell is selected from at least one point mutation, at least one insertion, or at least one deletion, or any combination thereof.

In one embodiment of the method for targeted modification of at least one genomic target sequence in a cell according to any of the embodiments described above, the cell is a eukaryotic cell, preferably a plant cell.

As demonstrated in the examples, the method is in particular applicable to plants, which opens new opportunities to improve crop traits.

In one embodiment of the method for targeted modification of at least one genomic target sequence in a cell according to any of the embodiments described above, the cell is a plant cell, which originates from a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Euca-lyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Gen-lisea aurea, Cucumis sativus, Morus notabilis, Arabidopsis arenosa, Arabidopsis lyrata, Ara-bidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medi-cago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Spinacia oleracea, Vicia faba, Phaseolus vulgaris, Allium cepa, Allium fistulosum, Allium sativum, and Allium tuberosum.

The present invention also relates to a cell preferably a eukaryotic cell, more preferably a plant cell obtainable by a method for targeted modification of at least one genomic target sequence according to any of the embodiments described above.

Furthermore, the present invention also relates to an organism, or part of an organism, preferably a plant or a part thereof, or a progeny thereof obtainable by cultivating a cell as described above, in particular a cell obtainable by a method for targeted modification of at least one genomic target sequence according to any of the embodiments described above.

Another way to expand or alter the PAM specificity of a given nuclease, it to exchange a domain of the nuclease with the corresponding domain of another nuclease, if the domain of the donor nuclease provides the desired PAM specificity and the nuclease remains overall functional.

The present invention therefore also provides a method of producing a chimeric MAD7-type nuclease, or a sequence encoding the same, the method comprising the following steps:
(a) defining the domain structure of a MAD7 nuclease and of at least one further CRISPR nuclease, wherein the different nucleases each have a defined PAM specificity and/or overall functionality;
(b) exchanging one defined domain from the MAD7 nuclease as recipient against one domain from the at least one further CRISPR nuclease as donor and thus creating a chimeric MAD7-type nuclease; and
(c) obtaining a chimeric MAD7-type nuclease;
(d) optionally: characterizing the chimeric MAD7-type nuclease of step (c).

In step (a) both nucleases are analysed structurally and functionally to define the domain structure and determine, which domains correspond to each other in the two nucleases and provide which PAM specificity. The nuclease with the desired PAM specificity then becomes the donor. The domain identified in step (a) of the donor is introduced into the recipient in step (b). The resulting chimeric nuclease then exhibits the PAM specific of the donor nuclease while retaining its overall structure and function.

In one embodiment of the method of producing a chimeric MAD7-type nuclease, the WED-II domain and PI domain of AsCpf1-RR (corresponding to amino acid positions 526-719 of SEQ ID NO: 47) is exchanged for the corresponding WED-II and PI domain in MAD7 (corresponding to amino acid positions 513-678 of SEQ ID NO: 42) resulting in MAD7-Cpf1 chimera I (SEQ ID NO: 40). Alternatively, amino acid positions 526-607 of SEQ ID NO: 47, including the WED-II, from AsCpf1-RR are exchanged for amino acid positions 513-594 of MAD7 (SEQ ID NO: 42) resulting in MAD7-Cpf1 chimera II (SEQ ID NO: 41).

The chimeric nucleases MAD7-Cpf1 chimera I and MAD7-Cpf1 chimera II show an increased activity at TYCV PAM sites compared to the MAD7 nuclease before domain exchange.

In order for the domain swap to result in a functional chimera, the two nucleases analyzed in step (a) are preferably related so that corresponding domains with the same functionality can be identified and swapped.

In one embodiment of the method of producing a chimeric MAD7-type nuclease, or a sequence encoding the same described above, the chimeric MAD7-type nuclease comprises at least one donor domain from a CRISPR class II nuclease, preferably from a CRISPR class II type V nuclease, more preferably from a CRISPR Cpf1/Cas12a nuclease.

The MAD7-type nuclease as defined in any of the embodiments above may also be used as a donor to transfer its PAM specificity to another nuclease.

The present invention therefore also provides a method of producing a chimeric nuclease, or a sequence encoding the same, the method comprising the following steps:
(a) defining the domain structure of a MAD7-type nuclease according to any of the embodiments described above, and of at least one further CRISPR nuclease, wherein the different nucleases each have a defined PAM specificity and/or overall functionality;
(b) exchanging one defined domain from the at least one further CRISPR nuclease as recipient against one domain from the MAD7-type nuclease as donor and thus creating a chimeric MAD7-type nuclease; and
(c) obtaining a chimeric nuclease;
(d) optionally: characterizing the chimeric nuclease of step (c).

In one embodiment of the method of producing a chimeric nuclease, or a sequence encoding the same, the MAD7-type nuclease has an engineered PAM specificity, wherein the nuclease is engineered to recognize at least one PAM selected from the group consisting of TYCV, TATV, or TTCN.

In one embodiment of the method of producing a chimeric nuclease, or a sequence encoding the same, the MAD7-type nuclease has a higher activity on a CTTN site in comparison to a MAD7 nuclease.

In one embodiment of the method of producing a chimeric nuclease, or a sequence encoding the same, the MAD7-type nuclease comprises at least one mutation at position 177, 272, 537, 543, 547, or 602 in comparison to the reference sequence of the MAD7 nuclease according to SEQ ID NO:3, or a combination of mutations.

In one embodiment of the method of producing a chimeric nuclease, or a sequence encoding the same, the at least one mutation described above is independently selected from K177R, N272A, D537R, K543V, K543R, N547R, K602R, or a combination thereof.

In one embodiment of the method of producing a chimeric nuclease, or a sequence encoding the same, the MAD7-type nuclease comprises a combination of mutations selected from D537R+K602R (MAD7-RR), D537R+K543V+N547R (MAD7-RVR), K177R+D537R (MAD7-V1), K177R+D537R+K543R (MAD7-V2), K177R+D537R+K602R (MAD7-RRR), K177R+D537R+K543V+N547R (MAD7-RRVR), K177R+D537R+N272A (MAD7-V1 + N272A), K177R+D537R+N272A+K543R (MAD7-V2 + N272A), K177R+D537R+N272A+K602R (MAD7-RRR + N272A), K177R+D537R+K543V+N272A+N547R (MAD7-RRVR + N272A).

In one embodiment of the method of producing a chimeric nuclease, or a sequence encoding the same, the MAD7-type nuclease comprises an amino acid sequence of any one of SEQ ID NOs: 12, 15, 18, 21, 24, 27, 30, 33, 36, 39, 40 or 41 or an amino acid sequence having at least 90%, 91%, 92%, 93%, 94%, 95%, 96%, 97%, 98% or 99% sequence identity to the respective amino acid sequence, or the nuclease is encoded by a nucleic acid sequence of any one of SEQ ID NOs: 10, 11, 13, 14, 16, 17, 19, 20, 22, 23, 25, 26, 28, 29, 31, 32, 34, 35, 37, 38 or a codon optimized variant thereof, or a nucleic acid sequence encoding an amino acid sequence having at least 90%, 91%, 92%, 93%, 94%, 95%, 96%, 97%, 98% or 99% sequence identity to any one of the respective SEQ ID NOs: 12, 15, 18, 21, 24, 27, 30, 33, 36, 39, 40 or 41.

As the MAD7-type nuclease of the present invention has a broad PAM specificity it can be used to perform genome editing processes in humans and animals. Thus, if a human or an animal carries a mutation in its genome, which mutation causes a disease, the MAD7-type nuclease of the present invention can be used to treat the disease.

The present invention also relates to a method of treating a disease in a subject, the method comprising the following steps:
(a) defining at least one mutation in the genome of a subject to be treated causing a disease;
(b) designing at least one guide nucleic acid sequence as defined above and optionally at least one repair template to modify the at least one mutation in a targeted way;
(c) introducing the MAD7-type nuclease as described in any of the embodiments above or the genome engineering system as described in any of the embodiments above or the expression construct as described in any of the embodiments above into at least one cell of a subject to be treated; and
(d) obtaining at least one cell comprising a targeted modification at the site of the at least one mutation causing a disease.

Once a mutation causing the disease is identified in the genome of the subject in step (a), one or more guide nucleic acid sequence(s) can be designed taking the available PAMs into account to target the mutation site or sites flanking the mutation. A MAD7-type nuclease according to the present invention can be chosen, which for example cuts out the mutation. The break can then be repaired using a repair template, which provides the sequence of a healthy subject.

In one embodiment of the method of treating a disease in a subject, the MAD7-type nuclease has an engineered PAM specificity, wherein the nuclease is engineered to recognize at least one PAM selected from the group consisting of TYCV, TATV, or TTCN.

In one embodiment of the method of treating a disease in a subject, the MAD7-type nuclease has a higher activity on a CTTN site in comparison to a MAD7 nuclease.

In one embodiment of the method of treating a disease in a subject, the MAD7-type nuclease comprises at least one mutation at position 177, 272, 537, 543, 547, or 602 in comparison to the reference sequence of the MAD7 nuclease according to SEQ ID NO:3, or a combination of mutations.

In one embodiment of the method of treating a disease in a subject, the at least one mutation described above is independently selected from K177R, N272A, D537R, K543V, K543R, N547R, K602R, or a combination thereof.

In one embodiment of the method of treating a disease in a subject, the MAD7-type nuclease comprises a combination of mutations selected from D537R+K602R (MAD7-RR), D537R+K543V+N547R (MAD7-RVR), K177R+D537R (MAD7-V1), K177R+D537R+K543R (MAD7-V2), K177R+D537R+K602R (MAD7-RRR), K177R+D537R+K543V+N547R (MAD7-RRVR), K177R+D537R+N272A (MAD7-V1 + N272A), K177R+D537R+N272A+K543R (MAD7-V2 + N272A), K177R+D537R+N272A+K602R (MAD7-RRR + N272A), K177R+D537R+K543V+N272A+N547R (MAD7-RRVR + N272A).

In one embodiment of the method of treating a disease in a subject, the MAD7-type nuclease comprises an amino acid sequence of any one of SEQ ID NOs: 12, 15, 18, 21, 24, 27, 30, 33, 36, 39, 40 or 41 or an amino acid sequence having at least 90%, 91%, 92%, 93%, 94%, 95%, 96%, 97%, 98% or 99% sequence identity to the respective amino acid sequence, or the nuclease is encoded by a nucleic acid sequence of any one of SEQ ID NOs: 10, 11, 13, 14, 16, 17, 19, 20, 22, 23, 25, 26, 28, 29, 31, 32, 34, 35, 37, 38 or a codon optimized variant thereof, or a nucleic acid sequence encoding an amino acid sequence having at least 90%, 91%, 92%, 93%, 94%, 95%, 96%, 97%, 98% or 99% sequence identity to any one of the respective SEQ ID NOs: 12, 15, 18, 21, 24, 27, 30, 33, 36, 39, 40 or 41.

The present invention also relates to a MAD7-type nuclease as described in any of the embodiments above, or the genome engineering system as defined in any of the embodiments described above or the expression construct as defined in any of the embodiments described above for use in a method of treating a disease in a subject.

Furthermore, the present invention also relates to a MAD7-type nuclease as described in any of the embodiments above, or the genome engineering system as defined in any of the embodiments described above or the expression construct as defined in any of the embodiments described above for modifying a genomic target site of interest, ex vivo or in vitro.

The present invention is further described with reference to the following non-limiting examples as well as the attached sequence listings and figures.

### Example 1: Codon Optimization of MAD7 Sequence for Expression in Zea mays

The E. coli optimized sequence of MAD7 was obtained from Inscripta Genomics Company and two versions were made through two different vendors for optimal corn expression and have been optimized for efficient expression through addition of NLS at N and C termini as well as addition of a translational enhancer at N terminus.

Version A of codon optimization (DNA: SEQ ID NO: 1; protein: SEQ ID NO: 3). Cloned into base vector and driven by Pol II promoter (BdUbi10; SEQ ID NO: 4). Resulting vector is pGEP837.

Version B of codon optimization (DNA: SEQ ID NO: 2; protein: SEQ ID NO: 3). Cloned into base vector and driven by Pol II promoter (BdUbi10; SEQ ID NO: 4). Resulting vector is pGEP838.

### Example 2: Expression of MAD7 Guide Sequence Flanked by Ribozyme Sequences From Pol II Promoter for Guide RNA Expression

A 35bp MAD7 scaffold sequence (SEQ ID NO: 5) was cloned into a base vector where it is flanked by a Hammerhead Ribozyme (SEQ ID NO: 6) at 5′ end and a HDV Ribozyme at 3′ end (SEQ ID NO: 7) all of which are driven by a ZmUbi1 promoter + intron (SEQ ID NO: 8 + SEQ ID NO: 9). Target guide sequences are cloned between the MAD7 scaffold and HDV ribozyme sequence by golden gate cloning and verified by sequencing.

### Example 3: Verification of MAD7 Activity at Target Sequences via Protoplast Assay and ddPCR and Next-Gen Sequencing

A two constructs combination consisting of a MAD7 nuclease expressing vector and a MAD7 target guide expression vector (pGEP842, pGEP846, pGEP843) are transformed into corn protoplasts (for detailed protocol see example 10 below) and after 24 h samples are collected and genomic DNA is extracted. For verifying MAD7 activity at three chosen targets, first a ddPCR assay was performed followed by NGS sequencing. ddPCR is designed according to Droplet Digital PCR Applications Guide from BioRad. The data (Table 2) indicates a higher efficiency of MAD7 activity (pGEP837 and pGEP838) at two target sites (crGEP5 and crGEP7) and comparable activity at the other target site (crGEP51) tested in plants over LbCpf1 (pGEP362; target guide expression vector: pGEP324, pGEP358, pGEP326).

TABLE 2

| Sample ID | Target | INDELS Ratio % | Protoplast Tfn Efficiency % | Normalized INDEL efficiency | Avg | std Dev |
|---|---|---|---|---|---|---|
| pGEP837+pGEP842-1 | crGEP5 | 16.3 | 21.5 | 75.7 | 70.0 | 6.5 |
| pGEP837+pGEP842-2 | | 14.1 | 19.8 | 71.2 | | |
| pGEP837+pGEP842-3 | | 11.7 | 18.5 | 63.0 | | |
| pGEP838+pGEP842-1 | crGEP5 | 12.7 | 20.7 | 61.3 | 55.8 | 5.7 |
| pGEP838+pGEP842-2 | | 12.0 | 23.9 | 50.0 | | |
| pGEP838+pGEP842-3 | | 12.1 | 21.7 | 56.0 | | |
| pGEP362+pGEP324-1 | crGEP5 | 9.2 | 22.5 | 41.0 | 47.3 | 5.6 |
| pGEP362+pGEP324-2 | | 10.2 | 20.5 | 49.7 | | |
| pGEP362+pGEP324-3 | | 11.9 | 23.2 | 51.4 | | |
| pGEP837+pGEP846-1 | crGEP51 | 3.0 | 19.5 | 15.3 | 16.3 | 1.4 |
| pGEP837+pGEP846-2 | | 3.1 | 19.7 | 15.6 | | |
| pGEP837+pGEP846-3 | | 3.5 | 19.8 | 17.9 | | |
| pGEP838+pGEP846-1 | crGEP51 | 2.4 | 22.3 | 10.8 | 14.6 | 3.2 |
| pGEP838+pGEP846-2 | | 3.9 | 23.6 | 16.5 | | |
| pGEP838+pGEP846-3 | | 3.4 | 20.5 | 16.5 | | |
| pGEP362+pGEP358-1 | crGEP51 | 3.7 | 21.9 | 17.0 | 16.9 | 1.9 |
| pGEP362+pGEP358-2 | | 4.3 | 23.0 | 18.8 | | |
| pGEP362+pGEP358-3 | | 3.6 | 24.1 | 14.9 | | |
| pGEP837+pGEP843-1 | crGEP7 | 8.8 | 24.8 | 35.7 | 41.6 | 5.3 |
| pGEP837+pGEP843-2 | | 8.7 | 18.8 | 46.0 | | |
| pGEP837+pGEP843-3 | | 11.7 | 27.2 | 43.0 | | |
| pGEP838+pGEP843-1 | crGEP7 | 8.6 | 23.1 | 37.2 | 39.9 | 4.1 |
| pGEP838+pGEP843-2 | | 8.3 | 21.9 | 37.8 | | |
| pGEP838+pGEP843-3 | | 12.0 | 26.9 | 44.6 | | |
| pGEP362+pGEP326-1 | crGEP7 | 0.9 | 21.5 | 4.3 | 4.8 | 1.4 |
| pGEP362+pGEP326-2 | | 1.0 | 26.1 | 3.8 | | |
| pGEP362+pGEP326-3 | | 1.6 | 25.4 | 6.5 | | |
| Untransformed control | crGEP5 | 0 | | | | |
| Untransformed control | crGEP51 | 0 | | | | |
| Untransformed control | crGEP7 | 0.1 | | | | |
| Water control | crGEp5 | 0 | | | | |
| Water control | crGEP7 | 0 | | | | |
| Buffer control | crGEP51 | 0 | | | | |
| Buffer control | crGEP7 | 0 | | | | |

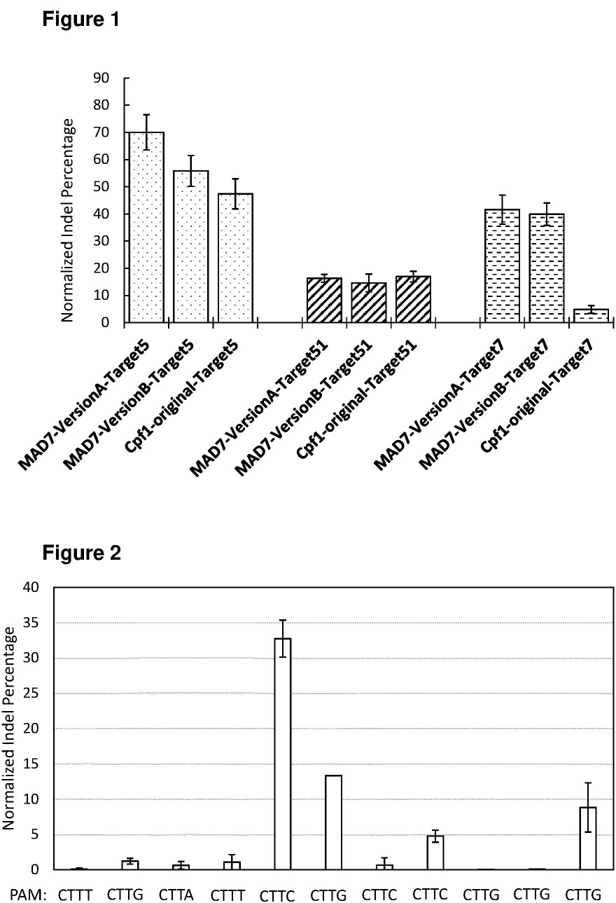

MAD7 can use CTTN PAM in corn protoplast. Codon optimized version A of MAD7 (from Genscript) was used in this experiment. Activity at CTTN PAM sites (FIG. 2) are demonstrated but not as high as on TTTN PAM sites (FIG. 1).

### Example 4: Engineering Change in PAM Preference of MAD7 From TTTN to TYCV and TATV

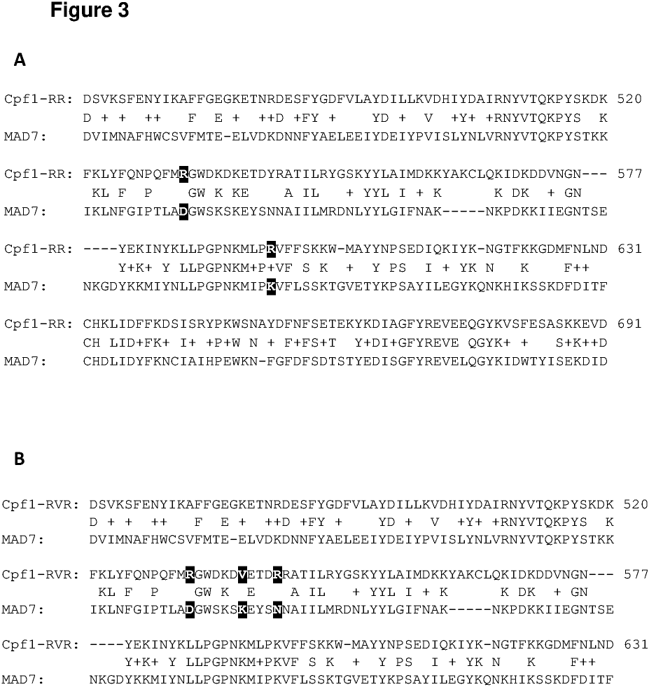

Alignment of protein sequence of LbCpf1 RR (SEQ ID NO: 44) and RVR (SEQ ID NO: 45) versions, which recognize TYCV and TATV PAMs, (the protein sequence of the LbCpf1 is given in SEQ ID NO:43) to MAD7 sequences identified conserved residues/regions where specific residues were changed to make RR (D529R and K594R) and RVR (D529R, K535V and N539R) versions of MAD7. Small synthetic DNA gBLOCKs were ordered and cloned into MAD7 sequences to change the specific amino acids (FIG. 3).

### Example 5: Verification of Activity at TYCV PAM Sites

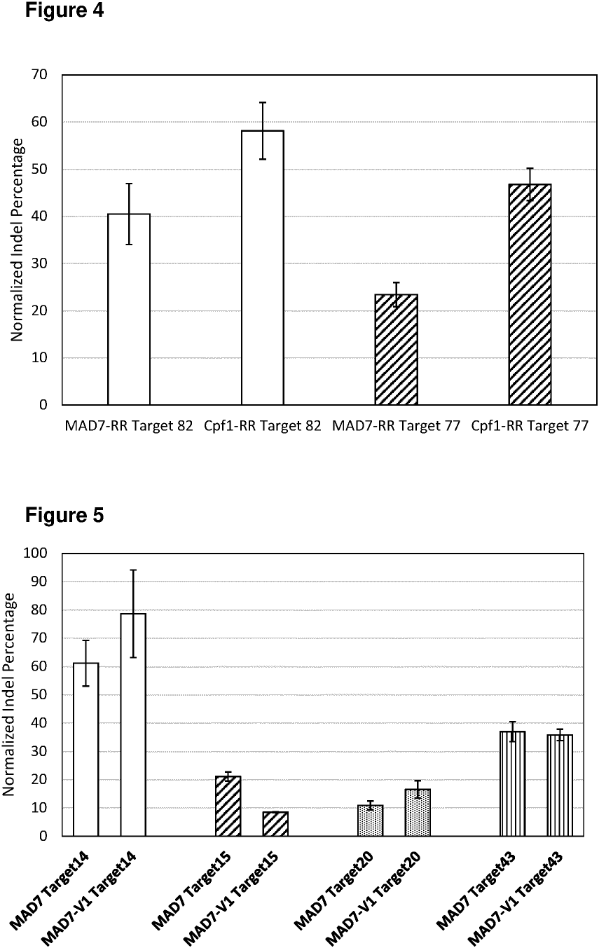

Two target sites, crGEP82 and crGEP77 were tested against MAD7-RR (SEQ ID NO: 12) and the original LbCpf1 RR (SEQ ID NO: 44) version in protoplast assays. Activity profile showed that activity of MAD7-RR is around 50-80% of LbCpf1-RR (see Table 3 and FIG. 4).

TABLE 3

| Sample ID | Nuclease | Target | INDELS Ratio % | Protoplast Tfn Efficiency % | Normalized INDEL efficiency | Avg | std Dev |
|---|---|---|---|---|---|---|---|
| GEP_PPLAST0042-v04-001 | MAD7-RR (Based on MAD7-Version A) | crGEP82 | 11.9 | 32.6 | 36.5 | 36.8 | 6.2 |
| GEP_PPLAST0042-v04-002 | | | 10.4 | 33.8 | 30.8 | | |
| GEP_PPLAST0042-v04-003 | | | 13.4 | 31.1 | 43.1 | | |
| GEP_PPLAST0042-v07-001 | LbCpf1 | crGEP82 | 16.1 | 37.2 | 43.3 | 40.8 | 3.6 |
| GEP_PPLAST0042-v07-003 | | | 6.9 | 18.1 | 38.2 | | |
| GEP_PPLAST0040-v07-001 | MAD7-RR (Based on MAD7-Version B) | crGEP82 | 8.1 | 31.4 | 25.9 | 20.8 | 4.5 |
| GEP_PPLAST0040-v07-002 | | | 5.0 | 29.2 | 17.3 | | |
| GEP_PPLAST0040-v07-003 | | | 5.2 | 27.0 | 19.3 | | |
| Untransformed control | | crGEP82 | 0.1 | | | | |
| Water control | | crGEP82 | 0 | | | | |

### Example 6: Introducing Other Mutations in MAD7 for Broader PAM Recognition and InCreased Activity

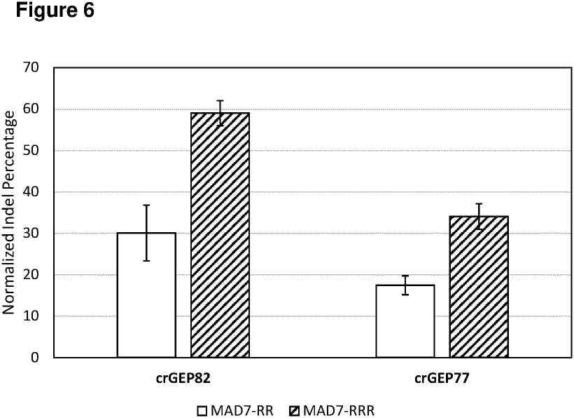

K177R and D537R were introduced into MAD7 (SEQ ID NO: 3) by changing the coding sequence from AAG to AGG (codon at nt positions 529-531 for K177R), GAC to CGC (codon at nt positions 1609-1611 for D537R) respectively in MAD7 codon optimized version. Site mutagenesis was used to introduce the mutation. Activity of the resulting MAD7-V1 (SEQ ID NO: 18) is tested against targets with YTTN and TTCN PAM sites. The results showed that the modified MAD7-V1 has similar or higher activity than the original MAD7 in three out of four CTTN targets tested (FIG. 5). MAD7-V2 (SEQ ID NO: 21) is generated by adding a third mutation (K543R) to MAD7-V1. The same site mutagenesis method is used to change the coding sequence (nt positions 1627-1629 in MAD7) from AAG to AGG for codon optimized version A and AAA to AGA for codon optimized version B at this site. Activity of the resulting MAD7-V2 is tested against targets with YTTN, TTCN and TATV PAM sites.

The activity of the modified MAD7-V1 (SEQ ID NO: 18) against targets with TTCN PAM were tested on 9 targets total from 2 different genes in corn protoplast. Results are shown in Table 4. High editing efficiency (above 20% after normalization with protoplast transformation efficiency) was found in 4 out of 9 total target sites.

TABLE 4

| Nuclease | Target | Normalized INDEL efficiency (%) | StError | PAM |
|---|---|---|---|---|
| MAD7-V1 | m7GEP91 | 0.69 | 0.17 | TTCA |
| MAD7-V1 | m7GEP92 | 0.25 | 0.01 | TTCG |
| MAD7-V1 | m7GEP93 | 20.68 | 3.90 | TTCT |
| MAD7-V1 | m7GEP94 | 0.23 | 0.23 | TTCT |
| MAD7-V1 | m7GEP96 | 0.00 | 0.00 | TTCG |
| MAD7-V1 | m7GEP97 | 25.99 | 6.92 | TTCA |
| MAD7-V1 | m7GEP98 | 59.25 | 8.46 | TTCA |
| MAD7-V1 | m7GEP99 | 0.10 | 0.02 | TTCG |
| MAD7-V1 | m7GEP100 | 32.72 | 4.09 | TTCA |
| MAD7-V1 | Controls | 0 | 0 | |

### Example 7: Combining K177R in MAD7-RR and MAD7-RVR Variants

The K177R mutation is introduced by converting AAG to AGG of the corresponding coding sequence. Small synthetic DNA gBLOCK are ordered and cloned into MAD7 sequences to change the specific amino acids. The resulting MAD7-RRR (SEQ ID NO: 24) and MAD7-RRVR (SEQ ID NO: 27) in both codon optimized versions are tested against targets with TYCV PAM or TATV PAM respectively. Two-times increase of activity on TYCV sites was observed when comparing MAD7-RRR with MAD7-RR (FIG. 6).

Table 5 shows data of MAD7-RRR (SEQ ID NO: 24) towards targets with TYCV PAM. Besides what has been already shown, the activity of the modified MAD7-RRR (SEQ ID NO: 24) against seven more targets with TYCV PAM from two different genes were tested in corn protoplast. Two out of 7 total sites were found with high editing efficiency (above 20% after normalization with protoplast transformation efficiency). Editing at target sites m7GEP104 and m7GEP107 were also tested with LbCpf1-RR in another experiment, where editing efficiency was found only at 19% and 9%, respectively. These results support that MAD7-RRR performs better than the original LbCpf1-RR in corn protoplast.

TABLE 5

| Nuclease | Target | Normalized INDEL efficiency (%) | StError | PAM |
|---|---|---|---|---|
| MAD7-RRR | m7GEP102 | 5.32 | 0.88 | TTCC |
| MAD7-RRR | m7GEP103 | 0.42 | 0.15 | TTCG |
| MAD7-RRR | m7GEP104 | 34.18 | 4.37 | TCCC |
| MAD7-RRR | m7GEP105 | 0.36 | 0.21 | TTCG |
| MAD7-RRR | m7GEP106 | 1.12 | 0.40 | TTCA |
| MAD7-RRR | m7GEP107 | 35.84 | 0.56 | TTCC |
| MAD7-RRR | m7GEP108 | 14.84 | 2.41 | TCCG |

### Example 8: Improving Specificity of MAD7 Variants

Using the same site mutagenesis method mentioned above, N272A (AAC -> GCC at nt positions 814 - 816) is introduced into MAD7 variants generated in example 6 and example 7. Off-targets of MAD7 variants with or without the N272A is compared using GUIDE-seq or Circle-Seq (Tsai et al. (2015). GUIDE-seq enables genome-wide profiling of off-target cleavage by CRISPR-Cas nucleases. Nature biotechnology, 33(2), 187.; Tsai et al. (2017). CIRCLE-seq: a highly sensitive in vitro screen for genome-wide CRISPR-Cas9 nuclease off-targets. Nature methods, 14(6), 607.).

### Example 9: Increasing MAD7 Activity on TYCV Sites by Domain Swapping

The whole WED-II domain and PI domain in AsCpf1-RR (amino acid position 526-719 of SEQ ID NO: 47) is amplified by PCR and used to replace the corresponding WED-II domain and PI domain (amino acid position 513-678 of SEQ ID NO: 42) in MAD7 in order to create MAD7-Cpf1 chimera I (SEQ ID NO:40). In another version (MAD7-Cpf1 chimera II; SEQ ID NO: 41), swap is performed at amino acid position 526-607 (including the WED-II domain) from AsCpf1-RR (SEQ ID NO: 47) to replace the amino acids position 513-594 in MAD7 (SEQ ID NO: 42). The resulting MAD7-Cpf1 chimeras are tested for activity on TYCV PAM sites. The protein sequence of AsCpf1 is given in SEQ ID NO: 46 and the protein sequence of the AsCpf1 RVR variant is given in SEQ ID NO: 48. Structure information on AsCpf1 is from Yamano et al., 2016 (Cell, Crystal Structure of Cpf1 in Complex with Guide RNA and Target DNA, 165(4): 949-62). Corresponding boundary of the domain in MAD7 is obtained by amino acid sequence alignment between AsCpf1 and MAD7.

### Example 10: Transformation of Corn Protoplasts

### Protocol

Add plasmid DNA (15 ug of the nuclease expressing plasmid plus 8 ug of the guide RNA expressing plasmid) to 2 ml tubes place at 4° C.
Harvest leaves from greenhouse of first and/ or second fully expanded true leaves from 10-14 day old etiolated seedlings.
Cut tissue into fine strips
Place cut tissue into deep petri dish with enzyme solution
Place into vacuum for 30 minutes.
Continue digestion for about 2 more hours on rocker at 28° C. in incubator.
Add equal amount of buffer and mix by gentle swirling.
Filter out tissue debris.
Put the protoplast solution through the filter.
Pellet cells at 100 g for 3 minutes at RT and remove supernatant.
Resuspend in buffer.
Centrifuge at 100 g for 2 minutes at RT and remove supernatant.
Resuspend in buffer to break up clumps and let cells settle for 30 minutes.
Remove supernatant from settled cells and resuspend pellet in adequate amount of MMG (0.4 M mannitol, 15 mM MgCl₂, pH 5.7).
Add 200 µl of resuspended protoplasts to each tube with DNA.
Add 220 µl of 40% PEG-CaCl₂ buffer and mix by tapping. Incubate for 5-10 minutes.
Stop the transfection with 880 µl of Stop Buffer and mix.
Centrifuge at 100 g for 2 minutes at RT. Remove supernatant.
Resuspend cells in 1 ml of buffer (e.g., W5 buffer).
Add 1 ml of buffer to 6-well plate and add the 1 ml of cells to the plate for a total of 2 ml.
Place in dark cabinet for 24 hours.

### Example 11: Multiplex Genome Editing in Corn Using MAD7-RRR

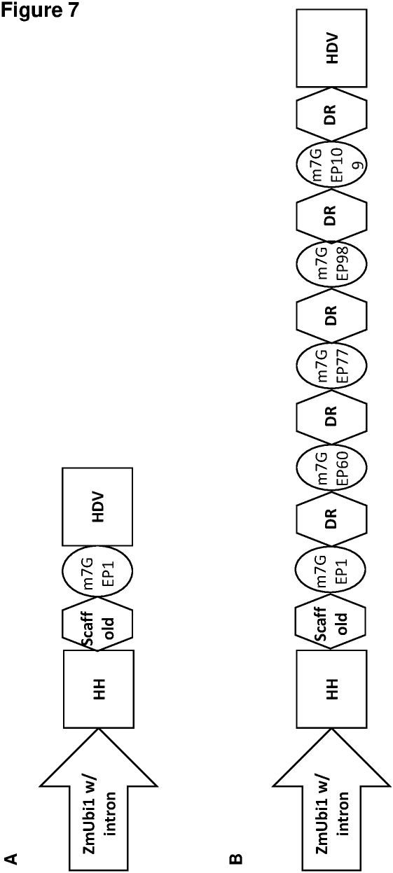

Multiplex genome editing was performed using the MAD7-RRR variant in corn to determine whether simultaneous genome editing at multiple different target sites can be achieved. Two strategies were used for guide RNA expression. In strategy 1, every individual guide RNA was expressed using the vector backbone created in Example 2 (FIG. 7A); whereas in strategy 2, multiple guide RNAs were expressed in a guide RNA array as demonstrated in FIG. 7B. Detailed information on all target sites and guide RNA expressing vectors used in multiplex editing experiments is listed in Table 6.

TABLE 6

| | | | | |
|---|---|---|---|---|
| Gene | Guide RNA ID | Construct Name | PAM | Guide RNA sequence |
| HMG13 | m7GEP1 | pGEP842 | TTTA | CTCGTCACGATTCCCCTCTCC (SEQ ID NO: 50) |
| | m7GEP2 | pGEP843 | TTTG | TGTGTGGTCACACTTGCCAGC (SEQ ID NO: 51) |
| ZmCPL3 | m7GEP60 | pGEZM006 | TTTG | GTCACTGCTGCCGGGGGCGGG (SEQ ID NO: 52) |
| | m7GEP64 | pGEZM010 | TTTC | AGGTGTCTGAGAAAACCAGTT (SEQ ID NO: 53) |
| Target III | m7GEP77 | pGEZM023 | TTTG | - |
| | m7GEP84 | pGEZM030 | CTTG | - |
| Target IV | m7GEP98 | pGEMT027 | TTCA | - |
| | m7GEP100 | pGEMT029 | TTCA | - |
| ZmCPL1 | m7GEP109 | pGEMT043 | TTTC | GAGGACAGAATTGATGCACTT (SEQ ID NO: 54) |
| | m7GEP113 | pGEMT047 | TTTA | GGTGGAGTACAGGTCAACATT (SEQ ID NO: 55) |

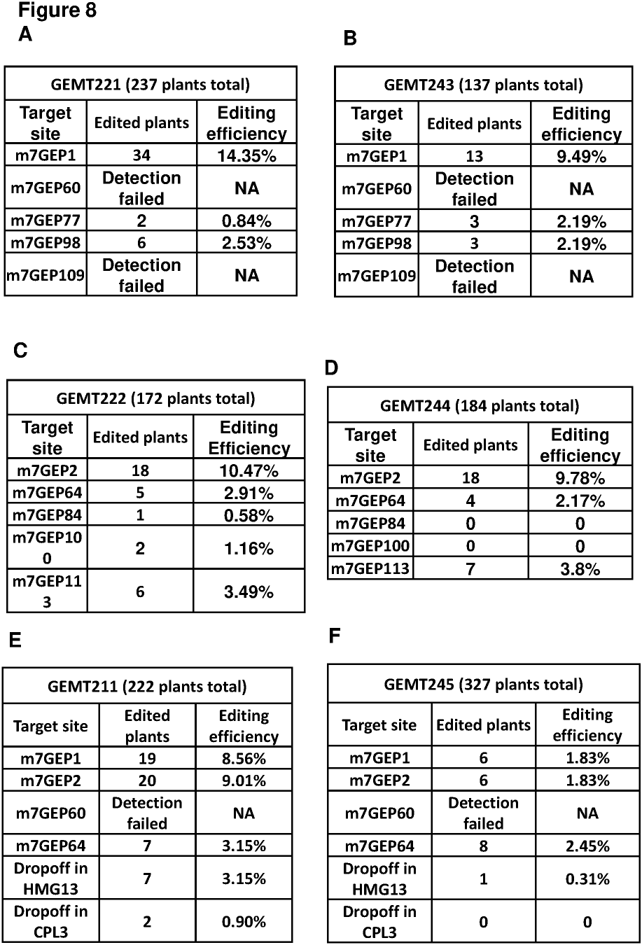

To test the possibility of simultaneous editing in 5 different genes, one target site in each gene was selected. In experiment GEMT221, plasmid constructs pGEP842, pGEZM006, pGEZM023, pGEMT027 and pGEMT043 (expressing m7GEP1, m7GEP60, m7GEP77, m7GEP98 and m7GEP109, respectively) together with the plasmid constructs expressing MAD7-V1, regeneration booster protein 2 (RBP2, SEQ ID NOs: 56 and 57) were co-bombarded into maize immature embryos (genotype A188) using biolistic delivery. In experiment GEMT243, a plasmid construct expressing a guide RNA array containing m7GEP1, m7GEP60, m7GEP77, m7GEP98 and m7GEP109 (FIG. 7B), together with the plasmid constructs expressing MAD7-V1, regeneration booster protein 2 were co-bombarded into maize immature embryos (genotype A188) using biolistic delivery. Individual plantlets were generated using the corn bombardment and regeneration protocol and editing at each target site was analyzed using qPCR and confirmed with sanger sequencing. Results of experiment GEMT221 and GEMT243 are shown in FIGS. 8A and 8B. In GEMT221, 8 plants out of 237 total plants were found edited at 2 target sites and 1 plant was found edited at 3 target sites. With the guide RNA array strategy, in GEMT243, 1 plant out of 137 total plants was found edited at 2 target sites. Another set of target sites (m7GEP2, m7GEP64, m7GEP84, m7GEP100, m7GEP113) targeting the same 5 genes as in experiment GEMT221 were used in the experiment GEMT222 and GEMT244, where guide RNAs were expressed as individual guide RNAs in GEMT222 and as a guide RNA array in GEMT244, respectively. Editing efficiency at each target site were shown in FIGS. 8C and 8D. Out of the 172 total plants in GEMT222, 2 plants were found edited at two target sites, 3 plants shown editing at three target sites and 2 plants were edited at all five target sites. In GEMT244, 6 plants were found edited at two target sites and 1 plant was edited at three target sites out of total 184 plants. These results demonstrate that multiplex editing in multiple different genes can be achieved with MAD7-V1 in corn.

Multiplex editing using two guide RNAs targeting the same gene is also performed. In both HMG13 and ZmCPL3 genes, one pair of target sites were selected (m7GEP1 and m7GEP2 in HMG13; m7GEP60 and m7GEP64 in CPL3) and guide RNAs targeting these sites were expressed either as individual guide RNA (in experiment GEMT211) or as a guide RNA array (in experiment GEMT245). As shown in FIGS. 8E and 8F, editing at individual target sites and simultaneous editing at two target sites resulting in DNA fragment drop off were detected in regenerated plants.

### Example 12: Corn Bombardment and Regeneration Protocol

### Step 1: Ear Sterilization

Maize ears with immature embryos size 0.5 to 2.5 mm were first sterilized with 10% bleach (8.25% sodium hypochlorite) plus 0.1% Tween 20 for 10 to 20 mins or 70% ethanol for 10-15 minutes and then washed four times with sterilized H₂O. Sterilized ears were dried briefly in a sterile hood for 5 to 10 mins.

### Step 2: Immature Embryos Isolation for Gold Particle Bombardment

Immature embryos of size, 0.6-2.0 mm, were isolated under sterile conditions by first removing the top third of the kernels from the ears with a sharp scalpel. Then immature embryos were carefully pulled out of the kernel with a spatula. The freshly isolated embryos were placed onto the bombardment target area in an osmotic medium plate (N6OSM-no2,4-D medium) with scutellum-side up. Plates were sealed and incubated at 25° C. in darkness for 4-20 hours before bombardment.

### Step 3: Bombardment

First, gold particles were prepared at a final concentration of sterile 50% (v/v) glycerol of 10 mg/ml. Then, DNA was coated onto the gold particles (for 10 bombardments) as follows: While vortex, the following has been added in order to each 100 µl of gold particles in 50% glycerol:
10 µl of DNA
100 µl of 2.5 M CaCl₂
40 µl of 0.1 M spermidine.

Allow the DNA-coated gold particles to settle 1 minute, spin for 5 seconds at the top speed, and then remove supernatant. The pellet was washed in 500 µl of 100% Ethanol for 1 minute and supernatant has been removed. Finally the DNA coated gold particles has been resuspended in 120 µl of 100% EtOH (for several bombardments).

In a next step, the gold particles were bombarded into the prepared immature embryos.

### Step 3: Post Bombardment Culture and Regeneration

First, the formation of Type II calli was induced 16-20 h post bombardment on a N6-5Ag plate with scutellum-face-up (at 27° C. in darkness for 14-16 days), before plants have been regenerated from the Type II callus.

### Media Used:

N6-5Ag: N6 salt + N6 vitamin + 1.0 mg/L of 2, 4-D + 100 mg/L of Caseine + 2.9 g/L of L-proline + 20 g/L sucrose + 5 g/L of glucose + 5 mg/L of AgNO3 + 8 g/L of Bacto-agar, pH 5.8

### Example 13: Testing of Sequence-Optimized MAD7 in Wheat Immature Embryos

This example establishes the sequence (codon) optimized MAD7 (GenScript optimized, pGEP837, Version A in Example 1) as a functional nuclease for use in wheat with CTTV and TTTV target sites.

Immature wheat embryos were isolated from donor plants and exposed to the MAD7 nuclease (Version A in Example 1) by particle bombardment. Individual guide RNA expressing vectors expressing guide RNAs that target different target sites was co-delivered with the constructs expressing MAD7. Target sites tested can be seen in FIGS. 9A and 9B. The embryos were harvested before regenerating into plants and analyzed by targeted amplicon sequencing for the specifically designed target sites.

During the analysis, all three wheat genomes were separated based on established SNPs. To correct for any inconsistencies during bombardment, a control experiment was performed at a well established target (TDF gene, Cas9 nuclease) that could be used to normalize the values obtained for the targets in the CPL3 gene. The average efficiency of the control target was 0.68%, 0.78%, and 0.66% for A, B, and D genome, respectively.

It was shown, that MAD7 is an active nuclease in wheat with activity across all genomes.

1. A nucleic acid guided nuclease, wherein the nuclease is a MAD7-type nuclease, or a sequence encoding the same, with an engineered PAM specificity, wherein the nuclease is engineered to recognize at least one PAM selected from the group consisting of TYCV, TATV, or TTCN.

2. The nucleic acid guided nuclease of claim 1, wherein the nuclease, or a domain thereof, comprises at least one mutation at position 177, 272, 537, 543, 547, or 602 in comparison to the reference sequence of the MAD7 nuclease according to SEQ ID NO:3, or a combination of mutations, in particular
wherein the at least one mutation is independently selected from K177R, N272A, D537R, K543V, K543R, N547R, K602R, or a combination thereof.

3. The nucleic acid guided nuclease of claim 1, wherein the nuclease, or a domain thereof, comprises a combination of mutations selected from D537R+K602R (MAD7-RR), D537R+K543V+N547R (MAD7-RVR), K177R+D537R (MAD7-V1), K177R+D537R+K543R (MAD7-V2), K177R+D537R+K602R (MAD7-RRR), K177R+D537R+K543V+N547R (MAD7-RRVR), K177R+D537R+N272A (MAD7-V1 + N272A), K177R+D537R+N272A+K543R (MAD7-V2 + N272A), K177R+D537R+N272A+K602R (MAD7-RRR + N272A), K177R+D537R+K543V+N272A+N547R (MAD7-RRVR + N272A).

4. The nucleic acid guided nuclease of claim 1, wherein the nuclease comprises an amino acid sequence of any one of SEQ ID NOs: 12, 15, 18, 21, 24, 27, 30, 33, 36, 39, 40 or 41 or an amino acid sequence having at least 90%, 91%, 92%, 93%, 94%, 95%, 96%, 97%, 98% or 99% sequence identity to the respective amino acid sequence, or wherein the nuclease is encoded by a nucleic acid sequence of any one of SEQ ID NOs: 10, 11, 13, 14, 16, 17, 19, 20, 22, 23, 25, 26, 28, 29, 31, 32, 34, 35, 37, 38 or a codon optimized variant thereof, or a nucleic acid sequence encoding an amino acid sequence having at least 90%, 91%, 92%, 93%, 94%, 95%, 96%, 97%, 98% or 99% sequence identity to any one of the respective SEQ ID NOs: 12, 15, 18, 21, 24, 27, 30, 33, 36, 39, 40 or 41.

5. The nucleic acid guided nuclease of claim 1, wherein the nuclease comprises at least one nuclear localization signal, preferably wherein the nuclease comprises one nuclear localization signal at the N-terminus and one nuclear localization signal at the C-terminus.

6. A genome engineering system comprising at least one MAD7-type nuclease of claim 1, or a sequence encoding the same, and at least one guide nucleic acid sequence, or a sequence encoding the same, wherein the at least one guide nucleic acid sequence comprises a scaffold region and a targeting region, preferably
wherein the targeting region targets a genomic target region of interest, which is an endogenous or isolated nucleic acid region of a eukaryotic or prokaryotic cell, in particular
wherein the genomic target region of interest is an endogenous or isolated nucleic acid region of a bacterial, fungal, animal, mammalian, or of a plant cell or organism.

7. The genome engineering system of claim 6, wherein the system additionally comprises at least one repair template, or a sequence encoding the same.

8. The genome engineering system of claim 6, wherein the at least one MAD7-type nuclease, or the sequence encoding the same, and/or the at least one guide nucleic acid, or the sequence encoding the same, and/or optionally the at least one repair template, or the sequence encoding the same, are provided simultaneously, or one after another.

9. An expression construct comprising or encoding at least one MAD7-type nuclease with an engineered PAM specificity, wherein the at least one MAD7-type nuclease is engineered to recognize at least one PAM selected from the group consisting of TYCV, TATV, or TTCN, and/or at least one guide nucleic acid sequence as defined in claim 6, and/or at least one repair template, preferably
wherein the construct comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

10. A kit comprising, in separate form, at least one compartment comprising at least one MAD7-type nuclease with an engineered PAM specificity, wherein the at least one MAD7-type nuclease is engineered to recognize at least one PAM selected from the group consisting of TYCV, TATV, or TTCN, or a sequence encoding the same, and optionally at least one guide nucleic acid sequence as defined in claim 6, or a sequence encoding the same, and optionally at least one repair template, or a sequence encoding the same, wherein the kit additionally comprises suitable reagents for each of the at least one compartment.

11. A method for the targeted modification of at least one genomic target sequence in a cell, wherein the method comprises the following steps:
(a) introducing into the cell
(i) at least one MAD7-type nuclease, or a sequence encoding the same, the at least one MAD7-type nuclease having an engineered PAM specificity, wherein the at least one MAD7-type nuclease is engineered to recognize at least one PAM selected from the group consisting of TYCV, TATV, or TTCN, and at least one guide nucleic acid sequence, or a sequence encoding the same, as defined in claim 6; or
(ii) at least one genome engineering system of claim 6 or at least one expression construct 9, wherein the at least one expression construct comprises at least one MAD7-type nuclease with an engineered PAM specificity, wherein the at least one MAD7-type nuclease is engineered to recognize at least one PAM selected from the group consisting of TYCV, TATV, or TTCN, and/or at least one guide nucleic acid sequence as defined in claim 6, and/or at least one repair template, and/or at least one regulatory sequence, and
(iii) optionally at least one repair template, or a sequence encoding the same;
(b) cultivating the cell under conditions allowing the expression and/or assembly of the genome engineering system comprising the at least one MAD7-type nuclease and the at least one guide nucleic acid sequence and optionally the at least one repair template; and
(c) obtaining at least one modified cell,
wherein (i), (ii), and optionally (iii) is/are introduced simultaneously or one after another and
wherein at least one of (i), (ii), and optionally (iii) is/are transiently introduced into and/or expressed in the cell or
wherein at least one of (i), (ii), and optionally (iii) is/are stably introduced into and/or expressed in the cell.

12. The method of claim 11, wherein at least two, three, four, five or more different guide nucleic acid sequences, or sequences encoding the same, are introduced into the cell to make multiple modifications in the cell simultaneously.

13. The method of claim 11, wherein the cell is a eukaryotic cell,
wherein the eukaryotic cell is a plant cell, which originates from a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Spinacia oleracea, Vicia faba, Phaseolus vulgaris, Allium cepa, Allium fistulosum, Allium sativum, and Allium tuberosum.

14. A cell, obtainable by a method of claim 11.

15. An organism, or part of an organism, or a progeny thereof obtainable by cultivating a cell of claim 14.


SEQUENCE LISTING (76 sequences)

SEQ ID NO: 1 - DNA, 3864 bp, Artificial Sequence
  Description of Artificial Sequence: Synthetic polynucleotide
  cDNA of codon-optimized MAD7 version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaagaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcggacggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 2 - DNA, 3864 bp, Artificial Sequence
  Description of Artificial Sequence: Synthetic polynucleotide
  cDNA of codon-optimized MAD7 version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaagaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatgaacctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctgacggctggtctaagagcaaagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 3 - protein, 1287 aa, Artificial Sequence
  Description of Artificial Sequence: Synthetic polypeptide
  MAD7 protein encoded by codon-optimzed MAD7 versions A and B
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 4 - DNA, 2503 bp, Brachypodium distachyon
ggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacag

SEQ ID NO: 5 - DNA, 35 bp, Artificial Sequence
  Description of Artificial Sequence: Synthetic oligonucleotide
  MAD7 scaffold sequence
gtcaaaagacctttttaatttctactcttgtagat

SEQ ID NO: 6 - DNA, 37 bp, Artificial Sequence
  Description of Artificial Sequence: Synthetic oligonucleotide
  Hammerhead Ribozyme
ctgatgagtccgtgaggacgaaacgagtaagctcgtc

SEQ ID NO: 7 - DNA, 68 bp, Artificial Sequence
  Description of Artificial Sequence: Synthetic oligonucleotide
  HDV Ribozyme at 3' end
ggccggcatggtcccagcctcctcgctggcgccggctgggcaacatgcttcggcatggcgaatgggac

SEQ ID NO: 8 - DNA, 987 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaag

SEQ ID NO: 9 - DNA, 520 bp, Zea mays
gtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 10 - DNA, 3864 bp, Artificial Sequence
  Description of Artificial Sequence: Synthetic polynucleotide
  cDNA of codon-optimized MAD7 RR version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaagaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgtggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgagggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 11 - DNA, 3864 bp, Artificial Sequence
  Description of Artificial Sequence: Synthetic polynucleotide
  cDNA of codon-optimized MAD7 RR version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaagaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatgaacctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgtggctggtctaagagcaaagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgagggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 12 - protein, 1287 aa, Artificial Sequence
  Description of Artificial Sequence: Synthetic polypeptide
  Protein MAD7 RR encoded by cDNA of codon-optimized MAD7 RR versions A and B
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSKEYSNNAIILMRDNLYYLGIFNAKNKPDKKIIEGNTSENKGDYKKMIYNLLPGPNKMIPRVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 13 - DNA, 3864 bp, Artificial Sequence
  Description of Artificial Sequence: Synthetic polynucleotide
  cDNA of codon-optimized MAD7 RVR version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaagaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgtggctggtccaagagcgtagagtactccaagaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 14 - DNA, 3864 bp, Artificial Sequence
  Description of Artificial Sequence: Synthetic polynucleotide
  cDNA of codon-optimized MAD7 RVR version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaagaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatgaacctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgtggctggtctaagagcgtagagtacagcaagaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 15 - protein, 1287 aa, Artificial Sequence
  Description of Artificial Sequence: Synthetic polypeptide
  Protein MAD7 RVR encoded by cDNA of codon-optimized MAD7 RVR versions A and B
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSVEYSRNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 16 - DNA, 3864 bp, Artificial Sequence
  Description of Artificial Sequence: Synthetic polynucleotide
  cDNA of codon-optimized mutated MAD7-V1 version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgcggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 17 - DNA, 3864 bp, Artificial Sequence
  Description of Artificial Sequence: Synthetic polynucleotide
  cDNA of codon-optimized mutated MAD7-V1 version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatgaacctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgcggctggtctaagagcaaagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 18 - protein, 1287 aa, Artificial Sequence
  Description of Artificial Sequence: Synthetic polypeptide
  Protein MAD7-V1 encoded by cDNA of codon-optimized MAD7-V1 versions A and B
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 19 - DNA, 3864 bp, Artificial Sequence
  Description of Artificial Sequence: Synthetic polynucleotide
  cDNA of codon-optimized mutated MAD7-V2 version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgcggctggtccaagagcagggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 20 - DNA, 3864 bp, Artificial Sequence
  Description of Artificial Sequence: Synthetic polynucleotide
  cDNA of codon-optimized mutated MAD7-V2 version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatgaacctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgcggctggtctaagagcagagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 21 - protein, 1287 aa, Artificial Sequence
  Description of Artificial Sequence: Synthetic polypeptide
  Protein MAD7-V2 encoded by cDNA of codon-optimized MAD7-V2 versions A and B
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSREYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 22 - DNA, 3864 bp, Artificial Sequence
  Description of Artificial Sequence: Synthetic polynucleotide
  cDNA of codon-optimized MAD7 RRR version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgtggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgagggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 23 - DNA, 3864 bp, Artificial Sequence
  Description of Artificial Sequence: Synthetic polynucleotide
  cDNA of codon-optimized MAD7 RRR version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatgaacctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgtggctggtctaagagcaaagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgagggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 24 - protein, 1287 aa, Artificial Sequence
  Description of Artificial Sequence: Synthetic polypeptide
  Protein MAD7 RRR encoded by cDNA of codon-optimized MAD7 RRR versions A and B
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSKEYSNNAIILMRDNLYYLGIFNAKNKPDKKIIEGNTSENKGDYKKMIYNLLPGPNKMIPRVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 25 - DNA, 3864 bp, Artificial Sequence
  Description of Artificial Sequence: Synthetic polynucleotide
  cDNA of codon-optimized MAD7 RRVR version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgtggctggtccaagagcgtagagtactccaagaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 26 - DNA, 3864 bp, Artificial Sequence
  Description of Artificial Sequence: Synthetic polynucleotide
  cDNA of codon-optimized MAD7 RRVR version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatgaacctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgtggctggtctaagagcgtagagtacagcaagaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 27 - protein, 1287 aa, Artificial Sequence
  Description of Artificial Sequence: Synthetic polypeptide
  Protein MAD7 RRVR encoded by cDNA of codon-optimized MAD7 RRVR versions A and B
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSVEYSRNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 28 - DNA, 3864 bp, Artificial Sequence
  Description of Artificial Sequence: Synthetic polynucleotide
  cDNA of codon-optimized mutated MAD7-V1 version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatggccctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgcggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 29 - DNA, 3864 bp, Artificial Sequence
  Description of Artificial Sequence: Synthetic polynucleotide
  cDNA of codon-optimized mutated MAD7-V1 version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatggccctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgcggctggtctaagagcaaagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 30 - protein, 1287 aa, Artificial Sequence
  Description of Artificial Sequence: Synthetic polypeptide
  Protein MAD7-V1 + N272A
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMALYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 31 - DNA, 3864 bp, Artificial Sequence
  Description of Artificial Sequence: Synthetic polynucleotide
  cDNA of codon-optimized mutated MAD7-V2 version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatggccctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgcggctggtccaagagcagggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 32 - DNA, 3864 bp, Artificial Sequence
  Description of Artificial Sequence: Synthetic polynucleotide
  cDNA of codon-optimized mutated MAD7-V2 version B
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatggccctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgcggctggtctaagagcagagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 33 - protein, 1287 aa, Artificial Sequence
  Description of Artificial Sequence: Synthetic polypeptide
  Protein MAD7-V2 + N272A
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMALYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSREYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 34 - DNA, 3864 bp, Artificial Sequence
  Description of Artificial Sequence: Synthetic polynucleotide
  cDNA of codon-optimized MAD7 RRR version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatggccctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgtggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgagggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 35 - DNA, 3864 bp, Artificial Sequence
  Description of Artificial Sequence: Synthetic polynucleotide
  cDNA of codon-optimized MAD7 RRR version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatggccctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgtggctggtctaagagcaaagagtacagcaacaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgagggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 36 - protein, 1287 aa, Artificial Sequence
  Description of Artificial Sequence: Synthetic polypeptide
  Protein MAD7 RRR + N272A
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMALYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSKEYSNNAIILMRDNLYYLGIFNAKNKPDKKIIEGNTSENKGDYKKMIYNLLPGPNKMIPRVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 37 - DNA, 3864 bp, Artificial Sequence
  Description of Artificial Sequence: Synthetic polynucleotide
  cDNA of codon-optimized MAD7 RRVR version A
  misc_feature (4)..(27): NLS
  misc_feature (3814)..(3861): NLS
atggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaggaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatggccctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcgcgtggctggtccaagagcgtagagtactccaagaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtga

SEQ ID NO: 38 - DNA, 3864 bp, Artificial Sequence
  Description of Artificial Sequence: Synthetic polynucleotide
  cDNA of codon-optimized MAD7 RRVR version B
  misc_feature (4)..(27): NLS
  misc_feature (3814)..(3861): NLS
atggccccgaagaagaagaggaaggtcaacaacggcaccaacaacttccagaacttcatcggcatcagcagcctgcaaaagaccctgaggaacgcgctgatcccgaccgagactacccagcagttcatcgtgaagaacggcatcatcaaagaggacgagctgaggggcgagaacaggcagatcctgaaggacatcatggacgactactacaggggcttcatcagcgagactctgagcagcatcgacgacatcgactggaccagcctgttcgagaagatggaaatccagctcaagaacggcgacaacaaggacaccctgatcaaagagcagaccgagtaccgcaaggccatccacaagaagttcgccaacgacgacaggttcaagaatatgttcagcgccaagctgatctccgacatcctgccagagttcgtgatccacaacaacaactacagcgccagcgagaaagaagaaaagacccaggtcatcaagctgttcagcaggttcgccaccagcttcaaggactacttcaggaacagggccaactgcttcagcgcggacgacatcagctctagcagctgccacaggatcgtgaacgacaacgccgagatcttcttcagcaacgccctggtgtacaggcgcatcgtcaagagcctgagcaacgacgatatcaacaagatcagcggcgacatgaaggacagcctcaaagagatgagcctggaagagatctacagctacgagaagtacggcgagttcatcacccaagagggcatcagcttctacaacgacatctgcggcaaggtgaactccttcatggccctgtactgccagaagaacaaagagaacaagaacctctacaagctgcagaagctgcacaagcagatcctctgcattgccgacacctcttacgaggtgccgtacaagttcgagagcgacgaagaggtgtaccagagcgtgaacggcttcctggacaacatctccagcaagcacatcgtcgagaggctgcgcaagatcggcgataactacaacggctacaacctcgacaagatctacatcgtgagcaagttctacgagagcgtgagccaaaagacctaccgcgactgggagactatcaacaccgcgctcgagatccattacaacaacatccttccaggcaacggcaagagcaaggccgacaaggtgaagaaggccgtcaagaacgacctgcagaagtccatcaccgagatcaacgagctggtgagcaactacaagctctgcagcgacgacaacatcaaggccgagacttacatccacgagatcagccacatcctgaacaacttcgaggcccaagagctgaagtacaacccagagatccacctggtcgagtccgagctgaaggcctctgagctgaagaacgtgctggacgtgatcatgaacgccttccactggtgcagcgtgttcatgaccgaggaactggtggacaaggataacaacttctacgccgagcttgaggaaatctacgacgagatctacccggtgatcagcctgtacaacctggtccgcaactacgtgacccagaagccgtacagcaccaagaagatcaagctgaacttcggcatcccgacgctggctcgtggctggtctaagagcgtagagtacagcaagaacgccatcatcctgatgagggacaacctctactacctgggcatcttcaacgccaagaacaagccggataagaagattatcgagggcaacacctccgagaacaagggcgactacaagaagatgatctataacctgctgccgggtccgaacaagatgatcccgaaggtgttcctgtccagcaagaccggcgtggaaacctacaagccgagcgcctacatcctcgagggctacaagcagaacaagcacattaagagcagcaaggacttcgatatcaccttctgccacgacctgatcgattactttaagaactgtatcgccatccatccagagtggaagaatttcggcttcgacttcagcgacaccagcacctacgaggacatcagcggtttctaccgcgaggttgagctgcaaggctacaagatcgattggacctacatctccgagaaggatatcgacctgctgcaagagaagggccagctgtacctgttccagatctacaacaaagacttcagcaagaagtccaccggcaacgacaacctgcacaccatgtacctgaagaacctgttcagcgaggaaaacctcaaggacatcgtgctgaagctgaacggcgaggcggagatattcttccgcaagtctagcatcaagaacccgatcatccacaaaaagggcagcatcctggtgaacaggacgtacgaggccgaggaaaaggaccagttcggcaacatccagatcgtgaggaagaacatcccagagaacatctaccaagagctttacaagtacttcaacgacaagtccgacaaagagctgagcgacgaggccgccaagcttaagaacgttgtgggccaccatgaggccgcgaccaacatcgttaaggactaccgctatacctacgataagtacttcctgcacatgccgatcaccatcaacttcaaggccaacaagaccggtttcatcaacgacaggatcctccagtacattgccaaagaaaaggatctgcacgttatcggcatcgacaggggcgagcgcaacctgatctacgtgtccgtgatcgatacctgcggcaacattgtcgagcaaaagtccttcaacatcgtgaatggctacgactaccagattaagctcaagcaacaagagggcgctcgccagatcgctaggaaagagtggaaagagatcggcaagatcaaagaaatcaaagagggctacctgagcctggtgatccatgagatctccaagatggttatcaagtacaacgcgatcattgccatggaagatctgtcctacggcttcaagaaaggccgcttcaaggtcgagcgccaggtctaccaaaagttcgaaaccatgctgatcaacaagctcaactacctcgtgttcaaggatatcagcatcactgagaacggcggcctgctgaagggctaccagctgacctacattccagacaagctgaaaaacgtcggccaccagtgcggctgcatcttctacgttccagccgcctacaccagcaagatcgacccaactaccggcttcgtgaacatcttcaagttcaaggacctgaccgtggacgccaagagggaattcatcaagaagtttgacagcatcaggtacgacagcgagaagaaccttttctgcttcaccttcgactacaacaactttatcacccagaacaccgtgatgagcaagagcagctggtccgtgtacacctacggcgtgaggatcaagcgccgcttcgttaacggcaggttctccaacgagtccgacaccatcgacatcaccaaggacatggaaaagactctcgagatgaccgacatcaactggcgcgacggccatgacctgaggcaggatatcatcgactacgagatcgtgcagcatatcttcgagattttcaggctgaccgtgcagatgaggaacagcctgagcgaactcgaggacagggactacgataggctcatcagcccggtgctgaacgagaacaacattttctacgactccgccaaggccggcgacgctttgccaaaagatgctgacgccaacggcgcctactgcattgctctgaagggcctgtacgagatcaagcagattaccgagaactggaaagaggatgggaagttctccagggataagctgaagatcagcaacaaggattggttcgatttcatccaaaacaagcgctacctcaagaggccagccgctaccaagaaggctggccaggcaaagaagaagaagtga

SEQ ID NO: 39 - protein, 1287 aa, Artificial Sequence
  Description of Artificial Sequence: Synthetic polypeptide
  Protein MAD7 RRVR + N272A
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMALYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSVEYSRNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 40 - protein, 1287 aa, Artificial Sequence
  Description of Artificial Sequence: Synthetic polypeptide
  MAD7-Cpf1 chimera I
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSVEKFKLNFQMPTLARGWDVNKEKNNGAILFVKNGLYYLGIMPKQKGRYKALSFEPTEKTSEGFDKMYYDYFPDAAKMIPRCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 41 - protein, 1287 aa, Artificial Sequence
  Description of Artificial Sequence: Synthetic polypeptide
  MAD7-Cpf1 chimera II
  SITE (2)..(9): NLS
  SITE (1272)..(1287): NLS
MAPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSVEKFKLNFQMPTLARGWDVNKEKNNGAILFVKNGLYYLGIMPKQKGRYKALSFEPTEKTSEGFDKMYYDYFPDAAKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKK

SEQ ID NO: 42 - protein, 1263 aa, Artificial Sequence
  Description of Artificial Sequence: Synthetic polypeptide
  MAD7
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 43 - protein, 1228 aa, Unknown
  Description of Unknown: Lachnospiraceae derived sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 44 - protein, 1228 aa, Artificial Sequence
  Description of Artificial Sequence: Synthetic polypeptide
  LbCpf1 RR
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 45 - protein, 1228 aa, Artificial Sequence
  Description of Artificial Sequence: Synthetic polypeptide
  LbCpf1 RVR
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 46 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 47 - protein, 1307 aa, Artificial Sequence
  Description of Artificial Sequence: Synthetic polypeptide
  AsCpf1 RR
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNKEKNNGAILFVKNGLYYLGIMPKQKGRYKALSFEPTEKTSEGFDKMYYDYFPDAAKMIPRCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 48 - protein, 1307 aa, Artificial Sequence
  Description of Artificial Sequence: Synthetic polypeptide
  AsCpf1 RVR
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 49 - DNA, 19 bp, Artificial Sequence
  Description of Artificial Sequence: Synthetic oligonucleotide
  Partial MAD7 scaffold sequence
aatttctactcttgtagat

SEQ ID NO: 50 - DNA, 21 bp, Artificial Sequence
  Description of Artificial Sequence: Synthetic oligonucleotide
  HMG13 guide RNA sequence 1
ctcgtcacgattcccctctcc

SEQ ID NO: 51 - DNA, 21 bp, Artificial Sequence
  Description of Artificial Sequence: Synthetic oligonucleotide
  HMG13 guide RNA sequence 2
tgtgtggtcacacttgccagc

SEQ ID NO: 52 - DNA, 21 bp, Artificial Sequence
  Description of Artificial Sequence: Synthetic oligonucleotide
  ZmCPL3 guide RNA sequence 1
gtcactgctgccgggggcggg

SEQ ID NO: 53 - DNA, 21 bp, Artificial Sequence
  Description of Artificial Sequence: Synthetic oligonucleotide
  ZmCPL3 guide RNA sequence 2
aggtgtctgagaaaaccagtt

SEQ ID NO: 54 - DNA, 21 bp, Artificial Sequence
  Description of Artificial Sequence: Synthetic oligonucleotide
  ZmCPL1 guide RNA sequence 1
gaggacagaattgatgcactt

SEQ ID NO: 55 - DNA, 21 bp, Artificial Sequence
  Description of Artificial Sequence: Synthetic oligonucleotide
  ZmCPL1 guide RNA sequence 2
ggtggagtacaggtcaacatt

SEQ ID NO: 56 - protein, 260 aa, Artificial Sequence
  Description of Artificial Sequence: Synthetic polypeptide
  RBP2 regeneration booster
MESGSGTAAGSGYVYRQSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNEDQTLIKSDKEIYCLGSCEKKMDLSPLGHSGSQRASALDLCLSLGNESCGLHDN

SEQ ID NO: 57 - DNA, 783 bp, Artificial Sequence
  Description of Artificial Sequence: Synthetic polynucleotide
  RBP2 regeneration booster coding sequence
atggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattga

SEQ ID NO: 58 - DNA, 21 bp, Artificial Sequence
  Description of Artificial Sequence: Synthetic oligonucleotide
  Target sequence 1 from Figure 9B
gtaccaggccaccagctaacc

SEQ ID NO: 59 - DNA, 21 bp, Artificial Sequence
  Description of Artificial Sequence: Synthetic oligonucleotide
  Target sequence 2 from Figure 9B
gctgaaggcttaagagcatgg

SEQ ID NO: 60 - DNA, 21 bp, Artificial Sequence
  Description of Artificial Sequence: Synthetic oligonucleotide
  Target sequence 3 from Figure 9B
gatcacactctcctcaattct

SEQ ID NO: 61 - DNA, 21 bp, Artificial Sequence
  Description of Artificial Sequence: Synthetic oligonucleotide
  Target sequence 4 from Figure 9B
aggacccagccatgctcttaa

SEQ ID NO: 62 - DNA, 21 bp, Artificial Sequence
  Description of Artificial Sequence: Synthetic oligonucleotide
  Target sequence 5 from Figure 9B
taggccacaagcaacatcgca

SEQ ID NO: 63 - DNA, 21 bp, Artificial Sequence
  Description of Artificial Sequence: Synthetic oligonucleotide
  Target sequence 6 from Figure 9B
gtacacggtcatcgctgtcaa

SEQ ID NO: 64 - DNA, 21 bp, Artificial Sequence
  Description of Artificial Sequence: Synthetic oligonucleotide
  Target sequence 7 from Figure 9B
ctcgtaccgacagaagtatgg

SEQ ID NO: 65 - DNA, 21 bp, Artificial Sequence
  Description of Artificial Sequence: Synthetic oligonucleotide
  Target sequence 8 from Figure 9B
agagcatggctgggtcctgaa

SEQ ID NO: 66 - DNA, 21 bp, Artificial Sequence
  Description of Artificial Sequence: Synthetic oligonucleotide
  Target sequence 9 from Figure 9B
gccttcctggaccatcacttc

SEQ ID NO: 67 - DNA, 21 bp, Artificial Sequence
  Description of Artificial Sequence: Synthetic oligonucleotide
  Target sequence 10 from Figure 9B
ttgtggggccagactctcaca

SEQ ID NO: 68 - DNA, 21 bp, Artificial Sequence
  Description of Artificial Sequence: Synthetic oligonucleotide
  Target sequence 11 from Figure 9B
attggaggggaagtggactgt

SEQ ID NO: 69 - DNA, 21 bp, Artificial Sequence
  Description of Artificial Sequence: Synthetic oligonucleotide
  Target sequence 12 from Figure 9B
agactactgtgttgccatctc

SEQ ID NO: 70 - DNA, 21 bp, Artificial Sequence
  Description of Artificial Sequence: Synthetic oligonucleotide
  Target sequence 13 from Figure 9B
catatgatggaatcggaacag

SEQ ID NO: 71 - DNA, 21 bp, Artificial Sequence
  Description of Artificial Sequence: Synthetic oligonucleotide
  Target sequence 14 from Figure 9B
gggggtgttgtatcagataat

SEQ ID NO: 72 - DNA, 21 bp, Artificial Sequence
  Description of Artificial Sequence: Synthetic oligonucleotide
  Target sequence 15 from Figure 9B
caagcccaaccccatctgatg

SEQ ID NO: 73 - protein, 231 aa, Artificial Sequence
  Description of Artificial Sequence: Synthetic polypeptide
DSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVD

SEQ ID NO: 74 - protein, 233 aa, Artificial Sequence
  Description of Artificial Sequence: Synthetic polypeptide
DVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDID

SEQ ID NO: 75 - protein, 171 aa, Artificial Sequence
  Description of Artificial Sequence: Synthetic polypeptide
DSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLND

SEQ ID NO: 76 - protein, 174 aa, Artificial Sequence
  Description of Artificial Sequence: Synthetic polypeptide
DVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITF